(19) United States
(12) Patent Application Publication
(10) Pub. No.: US 2018/0333042 A1
(43) Pub. Date: Nov. 22, 2018

(54) SELF-ANCHORING ENDOSCOPY SHEATH

(71) Applicant: Loubert S. Suddaby, Orchard Park, NY (US)

(72) Inventor: Loubert S. Suddaby, Orchard Park, NY (US)

(21) Appl. No.: 15/599,125

(22) Filed: May 18, 2017

(51) Int. Cl. A61B 1/00; A61B 17/02; A61B 1/018; A61B 1/313; A61B 1/015

(52) U.S. Cl. CPC A61B 1/00135; A61B 17/025; A61B 1/00154; A61B 1/018; A61B 2017/0256; A61B 1/00133; A61B 1/00066; A61B 1/00082; A61B 1/015; A61B 1/3135

(57) ABSTRACT

A self-anchoring endoscopy sheath, including a tubular member with a proximate end, a distal end, a through-bore, an inner surface, and an outer surface. The self-anchoring endoscopy sheath further includes at least one anchoring wire having a body and a tip, and at least one channel arranged between the inner surface and the outer surface, the at least one channel operatively arranged to receive and slidingly engage with the at least one anchoring wire wherein the tip of the anchoring wire is operatively arranged to engage with a tissue and the through-bore is operatively arranged to receive an endoscope.

### FIELD

The invention relates to spinal surgery, more particularly to endoscopic procedures, and, even more specifically, to an endoscopy sheath to be affixed to bony or soft tissue structures to maintain relative positioning during surgery such that both surgeon's hands are free to perform surgery.

### BACKGROUND

The spinal column, or backbone, is one of the most important parts of the body. It provides the main support, allowing us to stand upright, bend, and twist. As shown in FIG. 1, thirty three (33) individual bones interlock with each other to form the spinal column. The vertebrae are numbered and divided into regions. The cervical vertebrae (C1-C7) form the neck, support the head and neck, and allow nodding and shaking of the head. The thoracic vertebrae (T1-T12) join with the ribs to form the rib cage. The five lumbar vertebrae (L1-L5) carry most of the weight of the upper body and provide a stable center of gravity when a person moves. Five vertebrae of the sacrum S and four of the coccyx C are fused. This comprises the back wall of the pelvis. Intervertebral discs are located between each of the mobile vertebra. Intervertebral discs comprise a thick outer layer with a crisscrossing fibrous structure annulus that surrounds the soft gel-like center, the nucleus. Discs function like shock-absorbing springs. The annulus pulls the vertebral bodies together against the elastic resistance of the gel-filled nucleus. When we bend, the nucleus acts like a ball bearing, allowing the vertebral bodies to roll over the incompressible gel. Each disc works in concert with two facet joints, forming a spinal motion segment. The biomechanical function of each pair of facet joints is to guide and limit the movement of the spinal motion segment. The surfaces of the joint are coated with cartilage that helps each joint move smoothly. Directly behind the discs, the ring-like vertebral bodies create a vertical tunnel called the spinal canal or neuro canal. The spinal cord and spinal nerves pass through the spinal canal, which protects them from injury. The spinal cord is the major column of nerve tissue that is connected to the brain and serves as an information super-highway between the brain and the body. The nerves in the spinal cord branch off to form pairs of nerve roots that travel through the small openings between the vertebrae and the intervertebral foramens.

Various medical conditions require a surgeon to repair, remove and/or replace the aforementioned discs. For example, in one surgical procedure, known as a discectomy (or diskectomy), the surgeon removes the nucleus of the disk and replaces it with an implant. As shown in FIG. 2, it may be necessary, for example, for the surgeon to remove the nucleus of the disc between the L3 and L4 vertebrae. Disc D_{L3-L4} is shown in an enlarged view in FIG. 3. This figure also shows various anatomical structures of the spine, including facets F3A and F4A, facet joint FJ, spinous processes SP3 and SP4, transverse processes TP3A and TP4A, and intervertebral foramen IF. FIG. 4 is a top view of the section of the spinal column shown in FIG. 3, with the L3 vertebra removed to expose annulus A and nucleus N of disc D_{L3-L4}. Neural canal NC is also shown. FIG. 5 is an anterior perspective view of the section of the spinal column shown in FIG. 4. FIG. 6 is a partial cross-sectional view of the section of the spinal column shown in FIG. 5, but with vertebra L3 in place atop disc D_{L3-L4}.

One common tool used in these spinal surgical procedures is an endoscope. A representative endoscope 30 is shown in FIG. 7. Endoscopes are complex biomedical devices. The complexity results from the need for fiber optic bundles and multiple long narrow channels to be contained within a tubular structure that is constrained by the limited dimensions of the body cavity opening. As shown in FIG. 7, endoscope 30 broadly comprises light guide connector 31, light guide tube 32, control body 33, and insertion tube 34. As shown in FIG. 8, surgeon 40 uses the endoscope both to observe and guide the procedure via monitor 41, and to introduce and manipulate surgical instruments and tools during surgery on patient 45.

One problem associated with endoscopic surgery is holding the endoscope in position such that the surgeon has access to the enclosed working channels. Most endoscopic systems require the surgeon or an assistant to hold the endoscope while surgery is being performed, or employ crude and bulky mechanical arms to hold the endoscope in position.

In the event that the surgeon or assistant must hold the endoscope during surgery, arm fatigue makes it difficult to maintain the light source and working channel tip at the precise site where the surgical procedure is to be performed. Focus is frequently diverted from the procedure to repositioning the endoscope.

Therefore, there is a long-felt need for a self-anchoring endoscopic sheath that would maintain critical positioning of the distal portion of an endoscope such that illumination, magnification, and maintenance of the position of the distal working channel is assured.

### SUMMARY

According to aspects illustrated herein, there is provided a self-anchoring endoscopy sheath including a tubular member with a proximate end, a distal end, a through-bore, an inner surface, and an outer surface. The self-anchoring endoscopy sheath further includes at least one anchoring wire having a body and a tip, and at least one channel arranged between the inner surface and the outer surface, the at least one channel operatively arranged to receive and slidingly engage with the at least one anchoring wire wherein the tip of the anchoring wire is operatively arranged to engage with a tissue and the through-bore is operatively arranged to receive an endoscope.

According to aspects illustrated herein, there is provided a self-anchoring endoscopy sheath including a tubular member with a proximate end, a distal end, a through-bore, an inner surface, and an outer surface. The self-anchoring endoscopy sheath further includes a first expandable anchoring wire, and at least one channel arranged between the inner surface and the outer surface, the at least one channel operatively arranged to receive the first expandable anchoring wire wherein the first expandable anchoring wire is operatively arranged to engage with a tissue and the through-bore is operatively arranged to receive an endoscope.

According to aspects illustrated herein, there is provided a self-anchoring endoscopy sheath including a tubular member with a proximate end, a distal end, a through-bore, an inner surface, and an outer surface. The self-anchoring endoscopy sheath further comprises a first sealing member, a second sealing member, a cavity arranged between the first and second sealing members, at least one channel arranged between the inner surface and the outer surface, the at least one channel operatively arranged to connect a vacuum source to the cavity wherein the vacuum source generates a negative pressure within the channel and the cavity, securing the tubular member to a tissue via suction.

According to aspects illustrated herein, there is provided an endoscope assembly including an endoscope and a self-anchoring endoscopy sheath. The self-anchoring endoscopy sheath includes a tubular member having a proximate end, a distal end, a through-bore, an inner surface, and an outer surface. The self-anchoring endoscopy sheath also includes at least one anchoring wire having a body and a tip, and at least one channel arranged between the inner surface and the outer surface. The at least one channel is operatively arranged to receive and slidingly engage with the at least one anchoring wire and the tip of the anchoring wire is operatively arranged to engage with a tissue and the through-bore is operatively arranged to receive the endoscope.

It is intended that this device be applicable to all generally accepted surgical approaches to the spine, including microsurgical and endoscopic applications.

An additional object of the invention is to provide an assembly that can be built in or attached to an endoscope, or into a sheath or tube through which an endoscope is passed, such that the distal or working end position is fixedly maintained.

Another object of this invention is to free up both surgeon's hands and both assistant's hands so that the focus of their endeavors can be the surgery and not the maintaining the position of the endoscope.

It is a further object of this invention to readily permit polyaxial movement of the endoscope for superior viewing while maintaining a fixed position of the distal tip of the endoscope and working channel relative to the surrounding tissues.

To achieve these ends, an anchoring sheath is employed to fixate the distal tip at the preferred surgical site. It should be clearly understood that the various technologies here disclosed can be readily built into an endoscopic device or applied or affixed thereto. The preferred embodiment disclosed herein is mainly for ease of description of the invention and it should be appreciated that the sheath described permits the practice of the invention with any number of commercially available endoscopes already in use. It should be understood, therefore, that any reference to a sheath or tube could also reference an endoscope modified with the technology disclosed herein.

### BRIEF DESCRIPTION OF THE SEVERAL VIEWS OF THE DRAWINGS

The nature and mode of operation of the present disclosure will now be more fully described in the following detailed description of the embodiments taken with the accompanying figures, in which:

FIG. 1 is an anterior perspective view of spinal column 10;

FIG. 2 is an anterior perspective view of the lumbar section of spinal column 10;

FIG. 3 is a lateral perspective view of L3, L4 vertebrae and disc D_{L3-L4} and related spinal anatomy;

FIG. 4 is a top view of a section of the spinal column, taken generally along line 4-4 in FIG. 3;

FIG. 5 is an enlarged anterior perspective view of the spinal column shown in FIG. 2, except with vertebra L3 and all other structure above L3 removed;

FIG. 6 is a partial cross-sectional view of the L4 vertebra and D_{L3-L4} disc shown in FIG. 5, and also shows L3 in cross-section;

FIG. 7 is a perspective view of a typical endoscope;

FIG. 8 illustrates use of the endoscope shown in FIG. 7 by a surgeon performing a discectomy (diskectomy);

FIG. 9A is a side view of a non-threaded anchoring wire;

FIG. 9B is a side view of a partially-threaded anchoring wire;

FIG. 9C is a side view of a threaded anchoring wire;

FIG. 10 is a side perspective view of an endoscopy sheath;

FIG. 11A is a partial cross-sectional view, taken generally along line 11A-11A of FIG. 10, illustrating a non-threaded anchoring wire being introduced through an internal channel;

FIG. 11B is a partial cross-sectional view, taken generally along line 11B-11B of FIG. 10, illustrating a non-threaded anchoring wire that has traveled through the end of an internal channel, and has deflected in an outward radial direction;

FIG. 12 is a partial cross-sectional view, taken generally along line 12-12 of FIG. 10, illustrating multiple internal channels within an endoscopy sheath;

FIG. 13 is an enlarged view of box 13 in FIG. 11B illustrating the distal end of an endoscopy sheath;

FIG. 14A is a cross-sectional perspective view of an endoscopy sheath having external channels;

FIG. 14B is a cross-sectional perspective view of an endoscopy sheath having removable external channels;

FIG. 15A is a front cross-sectional view of the endoscopy sheath illustrated in FIG. 14A having external anchoring wire channels;

FIG. 15B is a front cross-sectional view of the endoscopy sheath illustrated in FIG. 14B having removable external anchoring wire channels;

FIG. 15C is a front cross-sectional view of an endoscopy sheath having embedded anchoring wire channels;

FIG. 16 illustrates a partial cross-sectional view of an impeller and an endoscopy sheath proximate to the disc with one anchoring wire introduced into vertebra L3;

FIG. 16A illustrates a partial cross-sectional view of the endoscopy sheath proximate to the disc with one anchoring wire introduced into vertebra L3;

FIG. 16B is an enlarged view of box 16B in FIG. 16A illustrating an endoscopy sheath proximate to the disc with one anchoring wire introduced into vertebra L3;

FIG. 17A illustrates a partial cross-sectional view of an endoscopy sheath proximate to the disc with two anchoring wires introduced into vertebrae L3 and L4;

FIG. 17B is an enlarged view of box 17B in FIG. 17A illustrating an endoscopy sheath proximate to the disc with two anchoring wires introduced into vertebrae L3 and L4;

FIG. 18A is a partial cross-sectional side view of an endoscopy sheath having a knurled wheel and an expandable anchoring wire with a first deformable section in a retracted state;

FIG. 18B is an enlarged view of box 18B in FIG. 18A illustrating a knurled wheel and its interaction with the threaded body of a threaded anchoring wire;

FIG. 18C is a partial cross-sectional view of an endoscopy sheath having a knurled wheel and an expandable anchoring wire with a first deformable section in an expanded state;

FIG. 19A is a partial cross-sectional view of the endoscopy sheath having a knurled wheel and an anchoring wire having first and second sections and a first hinge in a retracted state;

FIG. 19B is an enlarged view of box 19B in FIG. 19A illustrating a knurled wheel;

FIG. 19C is a partial cross-sectional view of the endoscopy sheath having a knurled wheel and an anchoring wire having first and second sections and a first hinge in an expanded state;

FIG. 20A is a partial cross-sectional view of the endoscopy sheath having a knurled wheel and an anchoring wire with first and second deformable sections in a retracted state;

FIG. 20B is an enlarged view of box 20B in FIG. 20A illustrating a knurled wheel;

FIG. 20C is a partial cross-sectional view of the endoscopy sheath having a knurled wheel and an anchoring wire with first and second deformable sections in an expanded state;

FIG. 21A is a partial cross-sectional view of the endoscopy sheath having a knurled wheel and first and second pivotable sections in a retracted state;

FIG. 21B is an enlarged view of box 21B in FIG. 21A illustrating a knurled wheel;

FIG. 21C is a partial cross-sectional view of the endoscopy sheath having a knurled wheel and first and second pivotable sections in an expanded state;

FIG. 22A is a partial cross-sectional view of the endoscopy sheath having a first inflatable balloon collar in a non-inflated state;

FIG. 22B is a partial cross-sectional view of the endoscopy sheath having a first inflatable balloon collar in an inflated state;

FIG. 23A is a partial cross-sectional view of the endoscopy sheath having first and second inflatable balloon collars in a non-inflated state;

FIG. 23B is a partial cross-sectional view of the endoscopy sheath having first and second inflatable balloon collars in an inflated state;

FIG. 24 is a partial cross-sectional view of the endoscopy sheath having a vacuum collar; and,

FIG. 25 is a side perspective view of the endoscope assembly having an endoscope operatively arranged within an endoscopy sheath.

### DETAILED DESCRIPTION OF THE INVENTION

At the outset, it should be appreciated that like drawing numbers on different drawing views identify identical, or functionally similar, structural elements of the invention. While the present invention is described with respect to what is presently considered to be the preferred aspects, it is to be understood that the invention as claimed is not limited to the disclosed aspect. The present invention is intended to include various modifications and equivalent arrangements within the spirit and scope of the appended claims.

The term “anchoring wire” as used in the present disclosure is intended to mean a substantially longitudinal wire used to penetrate soft or bony tissues and embed itself such that when embedded it will remain firmly in position maintaining the position of the endoscopy sheath. Suitable anchoring wires include but are not limited to K-wires or Kirschner wires widely used in orthopedics and other types of medicine. K-wires are well known in the art and have been used for temporary fixation during and after some surgical procedures.

The term “ratchet” as used in the present disclosure is intended to mean any mechanical device that allows continuous linear or rotary motion in only one direction while preventing motion in the opposite direction.

The term “balloon collar” as used in the present disclosure is intended to mean a substantially toroidal member arranged to receive a substance such as air, water, saline, or other equivalent material causing uniform expansion of the toroidal member.

The term “knurled wheel” as used in the present disclosure is intended to mean a hard or metallic wheel onto which indentations in the pattern of straight, angled, or crossed lines is cut or rolled onto the wheel to increase a user's ability to grip the wheel and displace it with a rotational force.

The term “impeller” as used in the present disclosure is intended to mean any mechanical device that imparts linear motion to at least one anchoring wire.

Furthermore, it is understood that this invention is not limited to the particular methodology, materials and modifications described and, as such, may, of course, vary. It is also understood that the terminology used herein is for the purpose of describing particular aspects only, and is not intended to limit the scope of the present invention, which is limited only by the appended claims.

Unless defined otherwise, all technical and scientific terms used herein have the same meaning as commonly understood to one of ordinary skill in the art to which this invention pertains. Although any methods, devices or materials similar or equivalent to those described herein can be used in the practice or testing of the invention, the preferred methods, devices, and materials are now described.

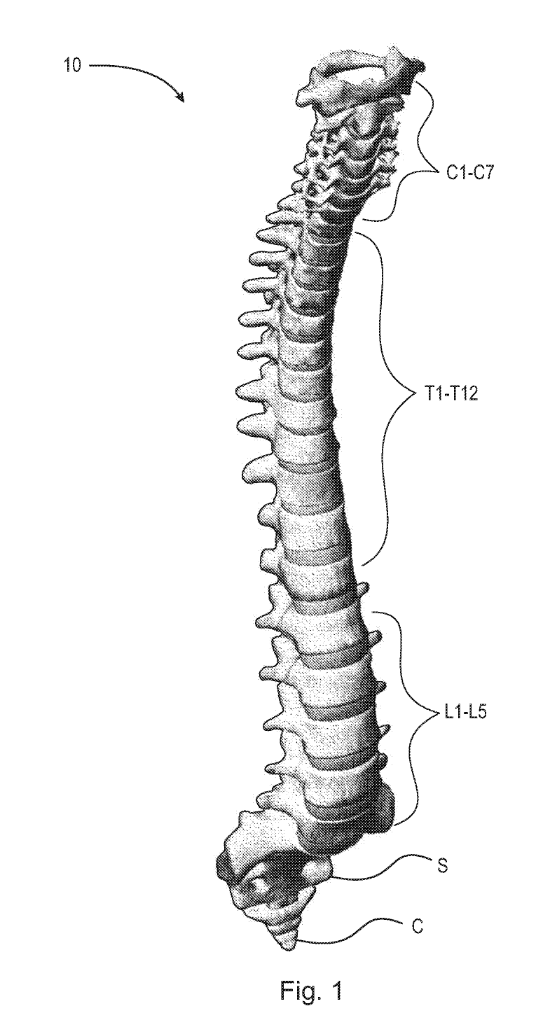

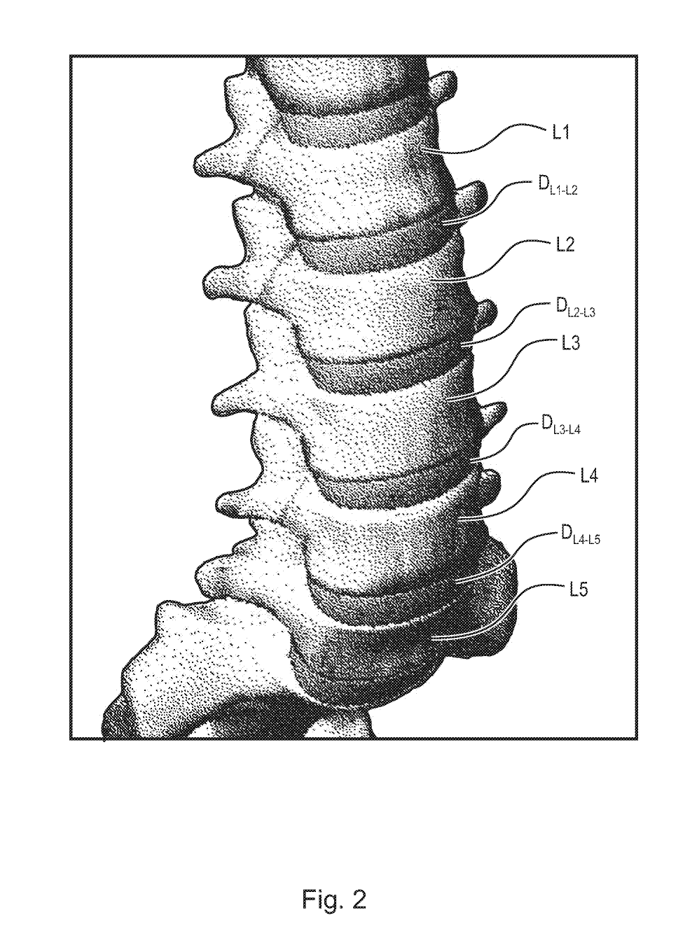

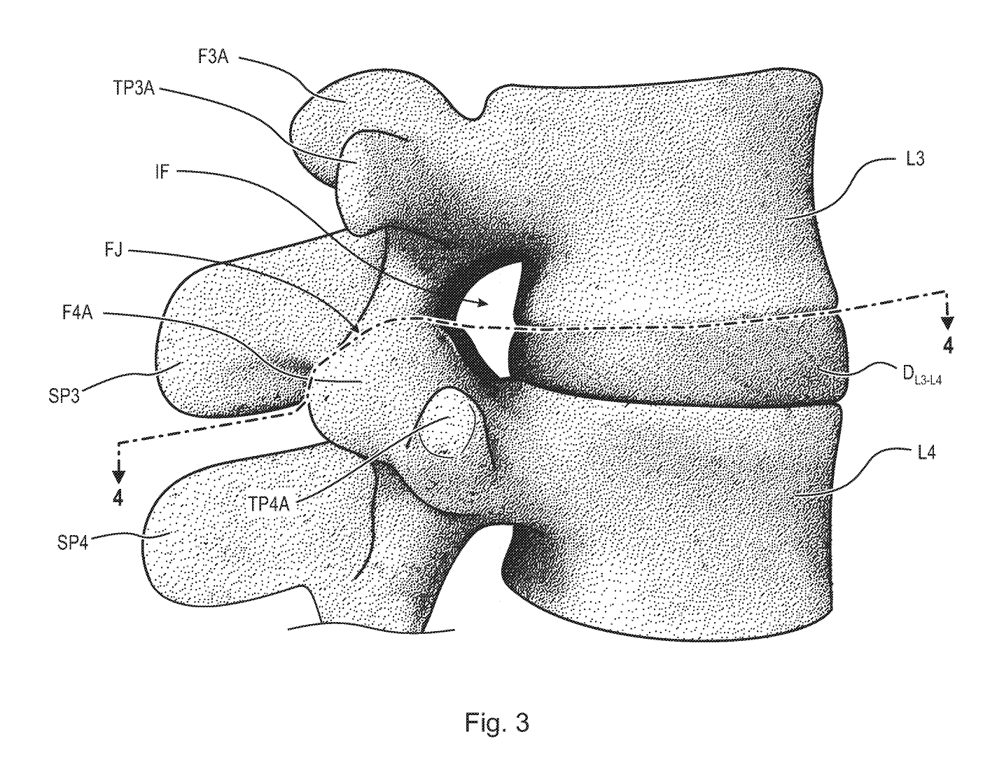

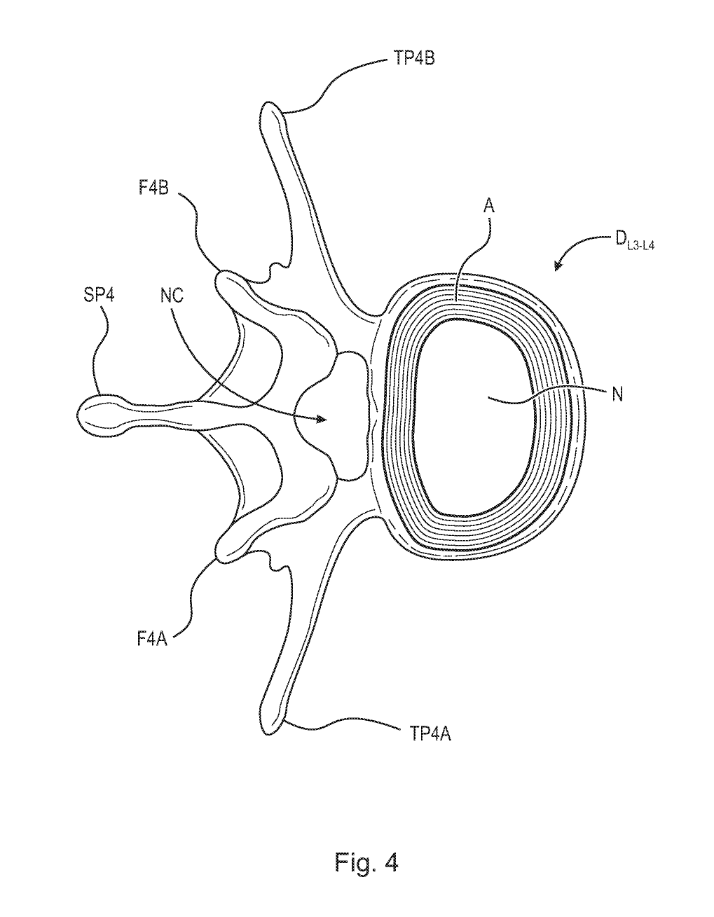

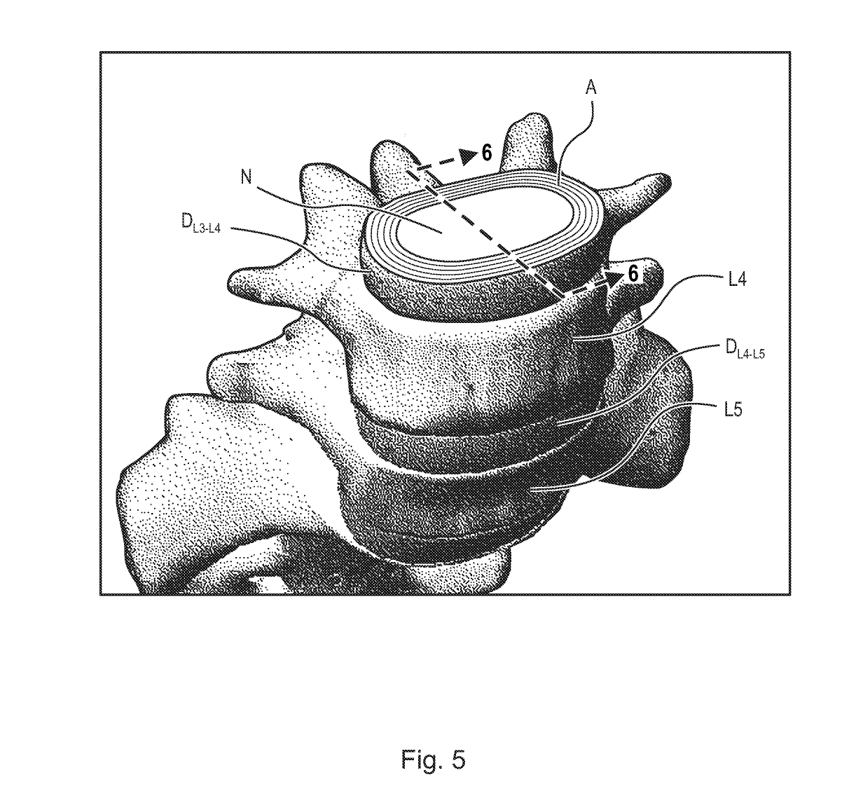

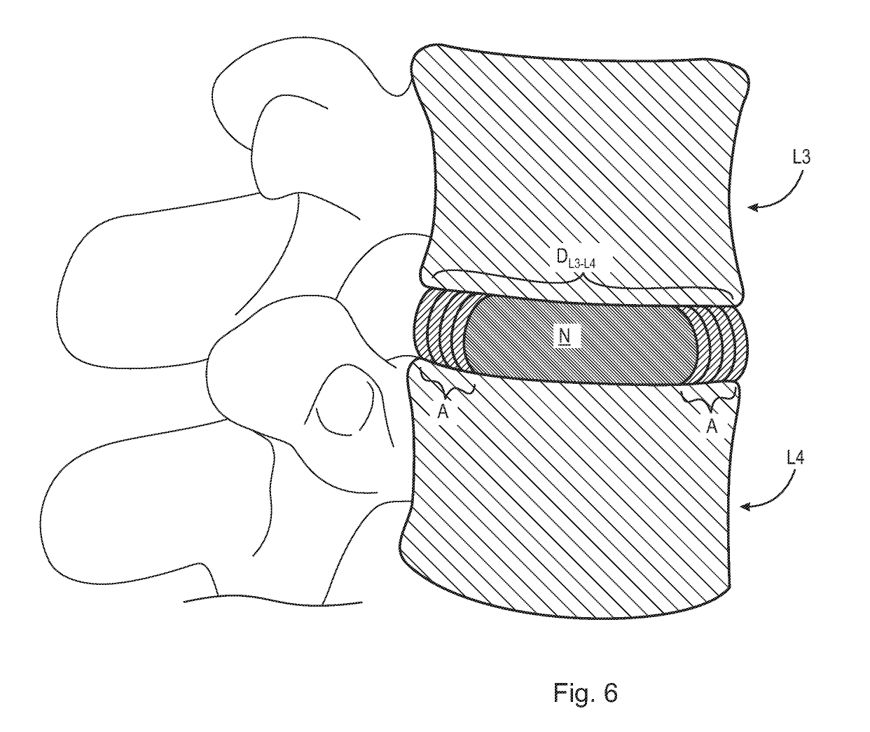

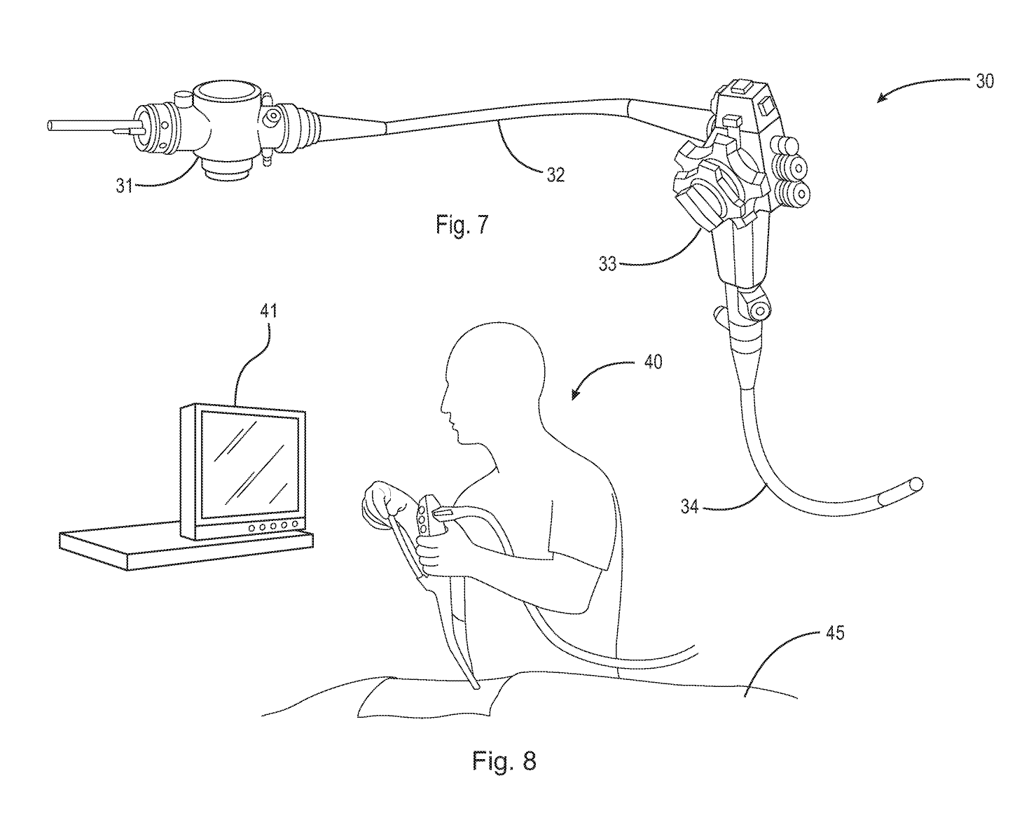

Adverting now to the Figures, and as described previously, FIGS. 1-6 depict various parts and sections of spinal anatomy, and FIGS. 7 and 8 depict a typical endoscope for use by surgeon 40 on a patient 45.

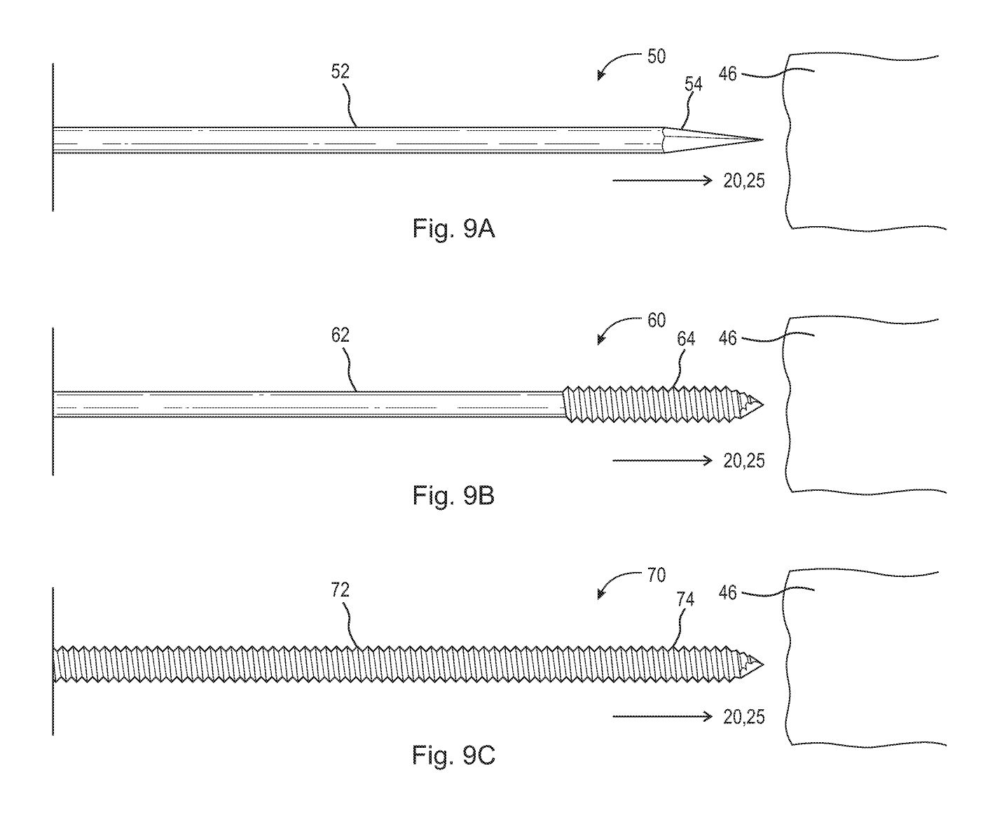

FIG. 9A is a side view of anchoring wire 50. Anchoring wire 50 comprises an unthreaded body 52, and an unthreaded tip 54 which is shown as a tapered point made of the same material as unthreaded anchoring wire body 52, e.g., stainless steel. It should be appreciated that although unthreaded tip 54 is illustrate as a pyramidal tapered point, other shapes can be utilized such as a conical tapered tip, or a frustoconical tip. Further, unthreaded body 52 is a substantially longitudinal member with a substantially circular cross section and an overall rod-like shape. During surgery, anchoring wire 50 can be inserted into a tissue 46 (either soft or bony) of patient 45 (shown in FIG. 8) by an imparted concussive force 20 or compressive force 25 which embeds the unthreaded tip 54 of anchoring wire 50 within tissue 46. It should be appreciated that compressive force 25 could be imparted manually via a single impact by a hammer or mallet as well as the continuous pressure imparted by a surgeon or assistant during surgery; or, in the case of concussive force 20, repeated impacts by a hammer or mallet.

FIG. 9B is a side view of anchoring wire 60. Anchoring wire 60 comprises an unthreaded body 62 and a threaded tip 64. Threaded Tip 64 is illustrated as a raised helical threaded portion operatively arranged to penetrate and twist into tissue 46. Similar to unthreaded body 52 discussed supra, unthreaded body 62 is a substantially longitudinal member with a substantially circular cross section and an overall rod-like shape. The twisting motion can be applied via impeller 124 discussed infra.

FIG. 9C is a side view of anchoring wire 70. Anchoring wire 70 comprises a threaded body 72 and a threaded tip 74. Threaded tip 74 and threaded body 72 are illustrated with a raised helical thread beginning at threaded tip 74 and continuing along the length of threaded body 72. The threaded tip 74 and threaded body 72 are operatively arranged to penetrate and twist into tissue 46. The twisting motion can be applied via impeller 124 or knurled wheel 132 discussed infra. Although not illustrated, it should be appreciated that a combination of a threaded body and an unthreaded tip could also be utilized.

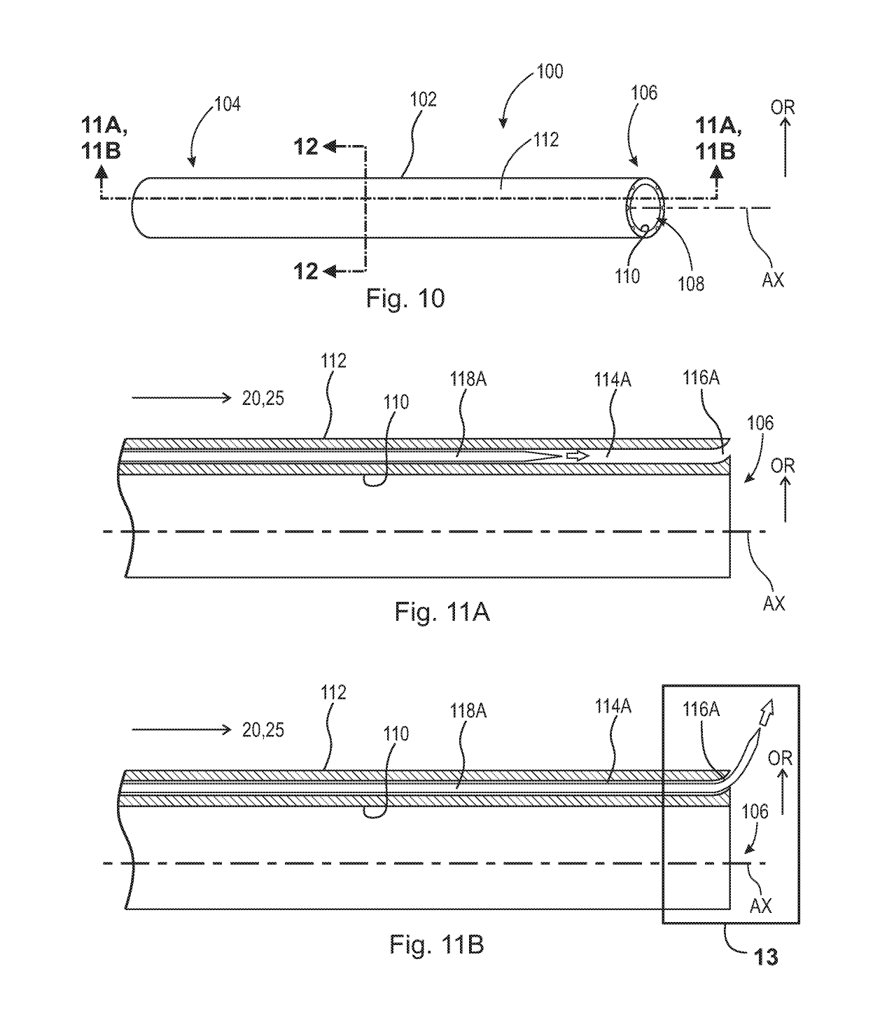

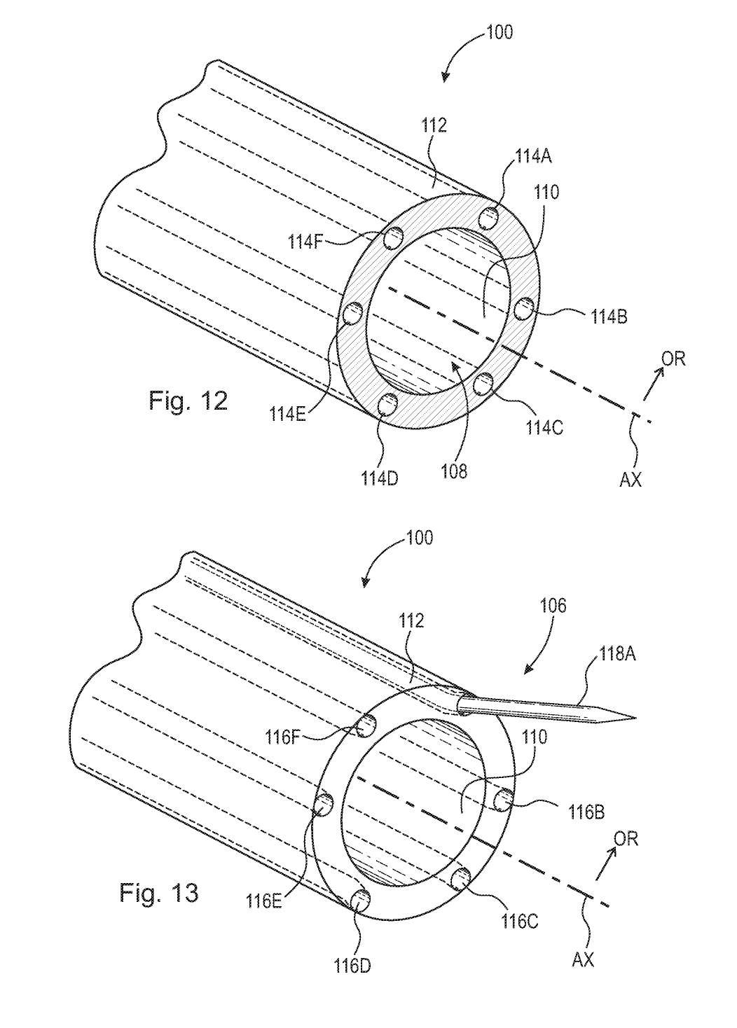

FIG. 10 is a side perspective view of endoscopy sheath 100. Endoscopy sheath 100 comprises a tubular member 102. Tubular member 102 comprises proximate end 104, distal end 106, a through-bore 108, an inner surface 110, and an outer surface 112. In a preferred embodiment tubular member 102 is made of stainless steel; however, other materials such as titanium or plastic can be utilized. Through-bore 108 is operatively arranged to receive and slidingly engage with endoscope 30. Inner surface 110 refers to the inner circumferential surface created by through-bore 108, and outer surface 112 refers to the outer circumferential surface of tubular member 102 (illustrated in FIGS. 12 and 13). Further, tubular member 102 comprises through-bore 108 which travels through tubular member 102, is concentric with, and is arranged about imaginary axis AX. Between inner surface 110 and outer surface 112 there are closed channels 114A-114F (illustrated in FIGS. 12 and 13). It should be appreciated that, although six (6) equally spaced channels are shown, any number of channels in any circumferentially spaced orientation can be utilized. Closed channels 114A-114F begin at proximate end 104 and terminate at distal end 106. Ramps 116A-116F are located within each of closed channels 114A-114F, respectively, proximate to the distal end 106 of tubular member 102. During surgery, a surgeon or assistant may insert anchoring wires 118A-118F into and along closed channels 114A-114F, respectively. Although illustrated as the embodiment of anchoring wire 50 having unthreaded body 52 and unthreaded tip 54, it should be appreciated that the embodiments of anchoring wire 60 and anchoring wire 70 could be utilized in this example embodiment.

FIG. 11A is a partial cross-sectional view, taken generally along line 11A-11A of FIG. 10, illustrating anchoring wire 118A being introduced through channel 114A. During surgery, it is desirable to maintain the position of the distal end 106 of tubular member 102 relative to a particular area within the human body, e.g., a human spine. To maintain such a position, a surgeon or assistant may hold endoscopy sheath 100 in the desired position, and subsequently insert anchoring wire 118A into any of channels 114A-114F. Using concussive force 20 or a compressive force 25, anchoring wire 118A is maneuvered along the length of a channel, e.g., channel 114A, until anchoring wire 118A reaches ramp 116A. It should be appreciated that although only anchoring wire 118A is discussed, the procedure described is applicable to anchoring wires 118A-118F. Further, anchoring wires 118A-118F can comprise the embodiments of anchoring wire 50, anchoring wire 60, or anchoring wire 70 in the discussed supra.

FIG. 11B is a partial cross-sectional view, taken generally along line 11B-11B of FIG. 10, illustrating anchoring wire 118A traveling through channel 114A. Upon contact with ramp 116A, anchoring wire 118A is diverted in outward radial direction OR. Outward radial direction OR is substantially orthogonal to axis AX. Diverting the body and tip of anchoring wire 118A in an outward radial direction OR prevents interference with endoscope 30, and provides for a stable anchoring point resistant to most forces applied to endoscopy sheath 100 during surgery.

FIG. 12 is a partial cross-sectional view, taken generally along line 12-12 of FIG. 10, illustrating channels 114A-114F within endoscopy sheath 100. FIG. 13 is an enlarged front perspective view of box 13 in FIG. 11B illustrating distal end 106 of an endoscopy sheath 100 along with the body and tip of anchoring wire 118A illustrating the diversion in outward radial direction OR after slidingly engaging ramp 116A.

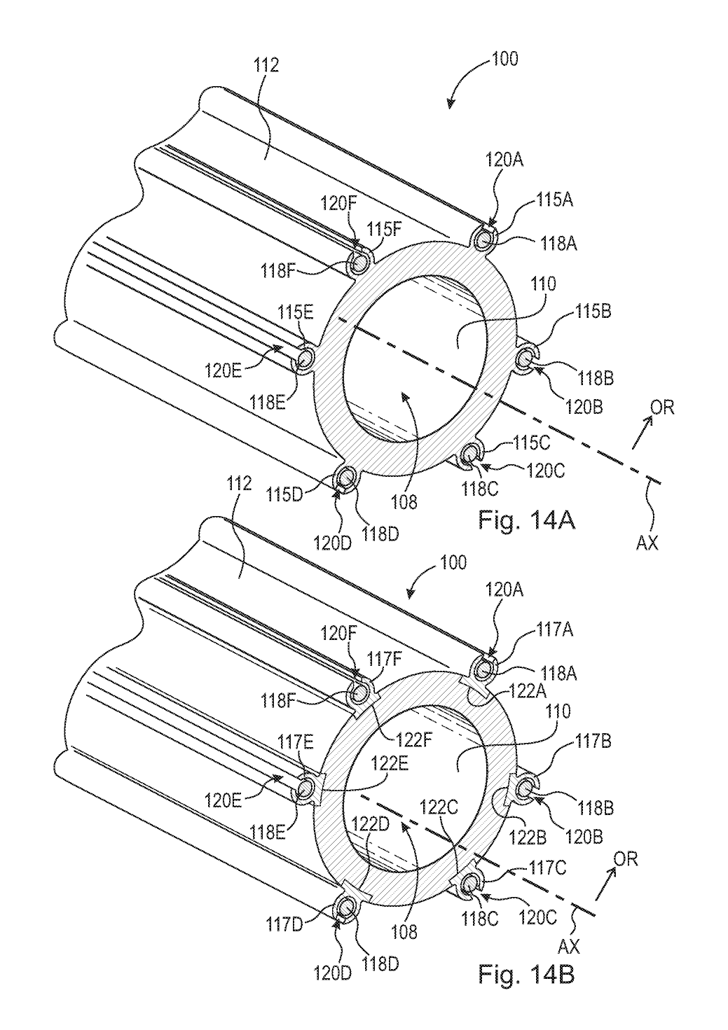

FIG. 14A is a cross-sectional perspective view of an example embodiment of endoscopy sheath 100 wherein semi-open external channels 115A-115F are arranged on outer surface 112. Each of semi-open external channels 115A-115F are arranged such that a substantial portion of the channel and anchoring wire are positioned in outward radial direction OR relative to outer surface 112. It should be appreciated that longitudinal spaces 120A-120F, which traverse the length of channels 115A-115F, allow the surgeon or assistant to view the progress of anchoring wires 118A-118F as they approach distal end 106 of endoscopy sheath 100. FIG. 14B is a cross-sectional perspective view of an example embodiment of endoscopy sheath 100 wherein semi-open external removable channels 117A-117F are arranged on the outer surface 112 and are removable. Semi-open external removable channels 117A-117F are arranged to slidingly engage with recesses 122A-122F, respectively. It should be appreciated that longitudinal spaces 120A-120F, which traverse the length of channels 117A-117F, allow the surgeon or assistant to view the progress of anchoring wires 118A-118F as they approach distal end 106 of endoscopy sheath 100.

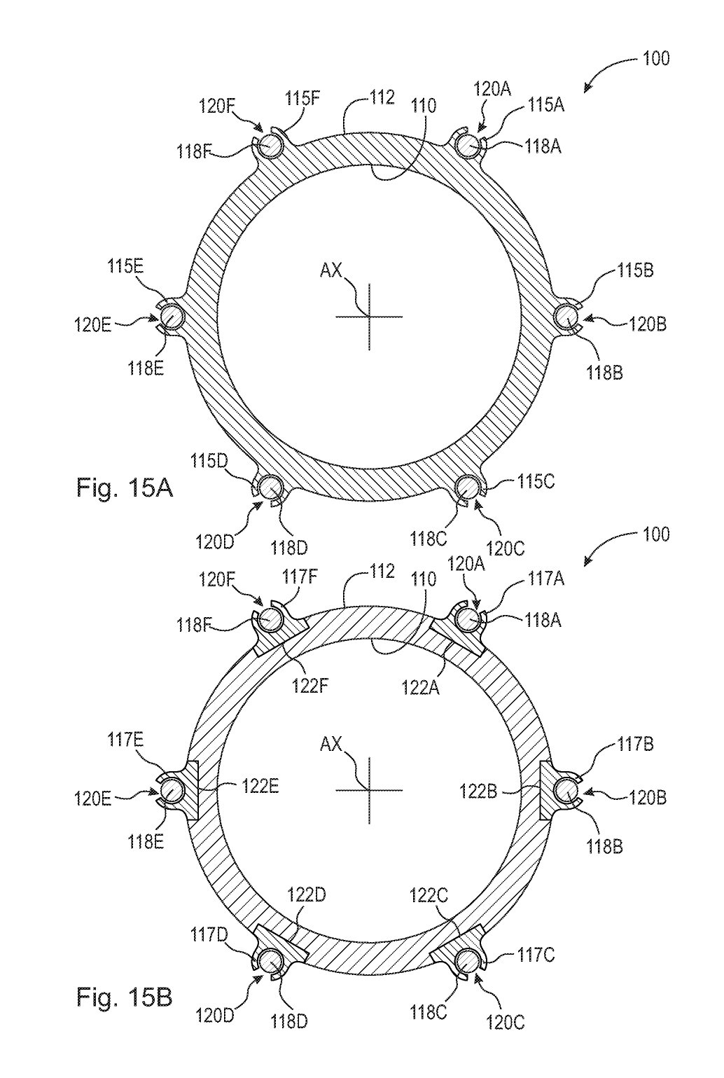

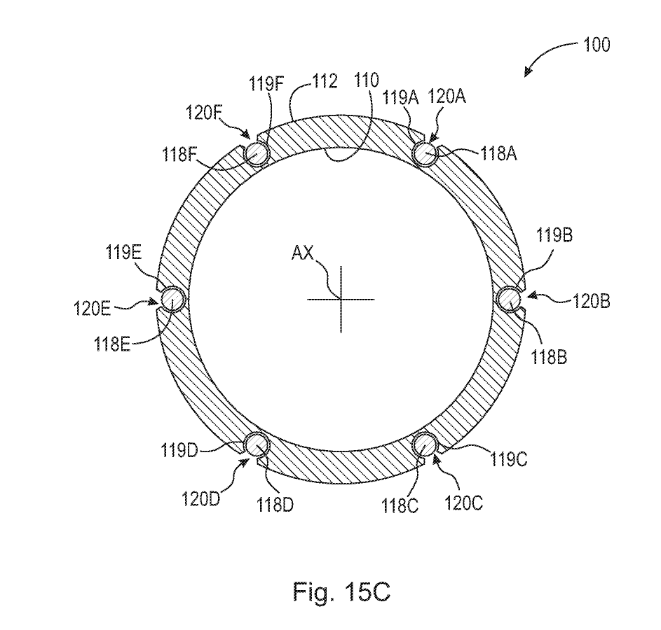

FIG. 15A is a front cross-sectional view of endoscopy sheath 100 illustrated in FIG. 14A wherein semi-open external channels 115A-115F are arranged on the outer surface 112 of endoscopy sheath 100 instead of between outer surface 112 and inner surface 110. FIG. 15B is a front cross-sectional view of endoscopy sheath 100 illustrated in FIG. 14B wherein semi-open external removable channels 117A-117F are arranged on the outer surface 112 and are removable. FIG. 15C is a front cross-sectional view of an example embodiment of endoscopy sheath 100 having semi-open internal channels 119A-119F that are positioned between outer surface 112 and inner surface 110, where outer surface 112 comprises longitudinal spaces 120A-120F arranged radially outward with respect to semi-open internal channels 119A-119F.

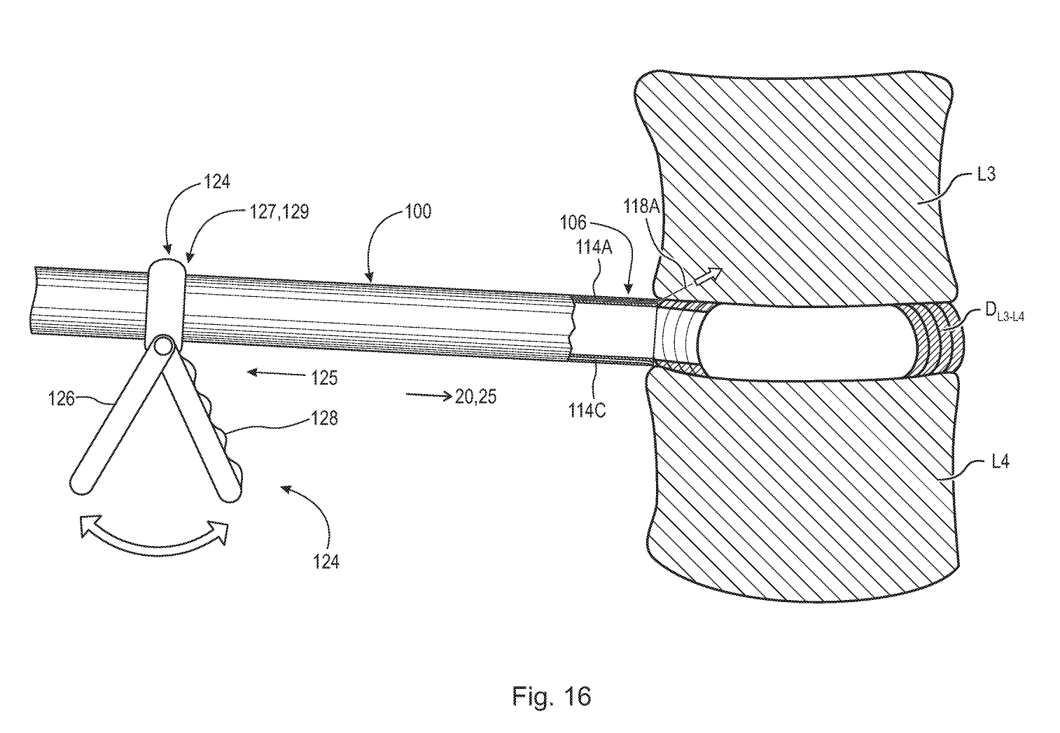

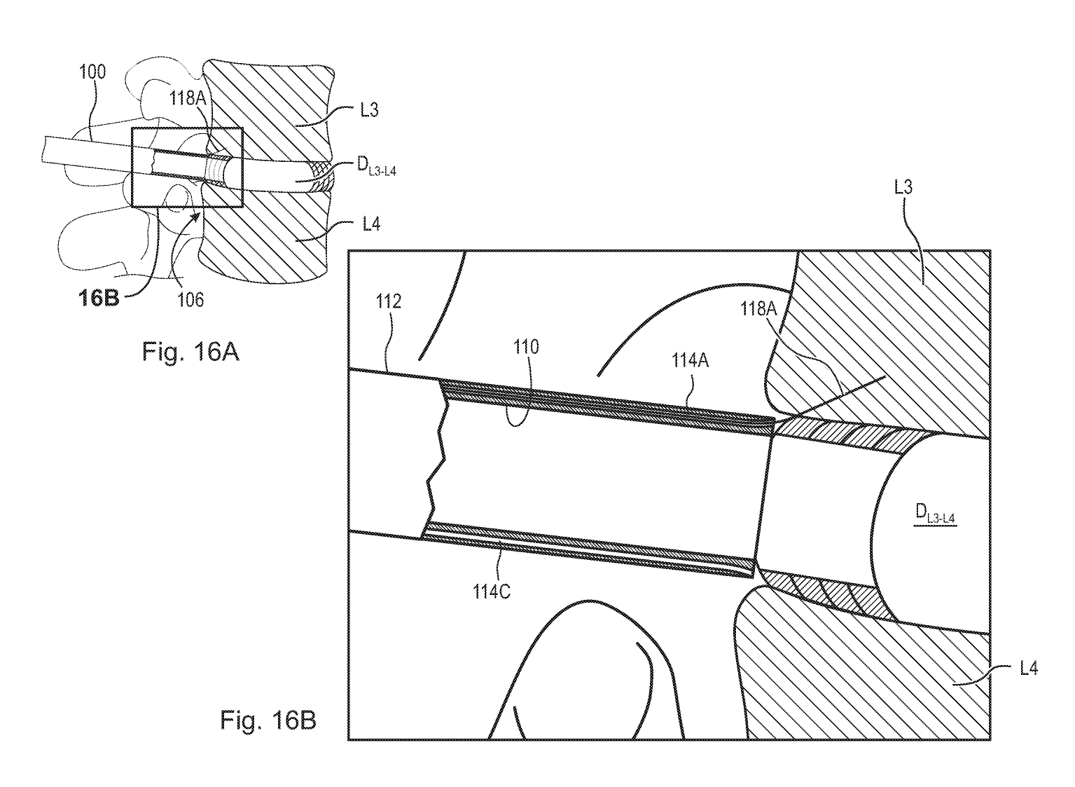

FIG. 16 illustrates a partial cross-sectional view of impeller 124 and endoscopy sheath 100 proximate to disc D_{L3-L4} with anchoring wire 118A introduced into vertebra L3. During surgery, a surgeon or assistant may utilize impeller 124 to impart a compressive force 25 or a concussive force 20 on anchoring wires 118A-118F. Impeller 124 comprises pivot 125, handle 126, first ring 127, lever 128, and second ring 129. First ring 127 is pivotably secured to handle 126 and fixedly secured to outer surface 112 of tubular member 102. Second ring 129 is pivotably secured to lever 128 and anchoring wires 118A-118F. During surgery, a surgeon or assistant may squeeze together handle 126 and lever 128. Handle 126 and lever 128 rotate around pivot 125. Since handle 126 is pivotably secured to first ring 127, handle 126 acts as a stationary anchor point. As lever 128 rotates about pivot 125, anchoring wires 118A-118F translate with second ring 129 towards distal end 106 of tubular member 102. It is important to note that during surgery it may be desirable to use only one anchoring wire, e.g., anchoring wire 118A, so as to allow maximum range of pivotable motion of endoscopy sheath 100, while also keeping distal end 106 of endoscopy sheath 100 in the same general position. Although it is not illustrated, it is possible to arrange impeller 124 with a stopping mechanism, e.g., a ratchet which prevents motion in one direction while allowing motion in the opposite direction keeping anchoring wires 118A-118F in place once they enter tissue 46.

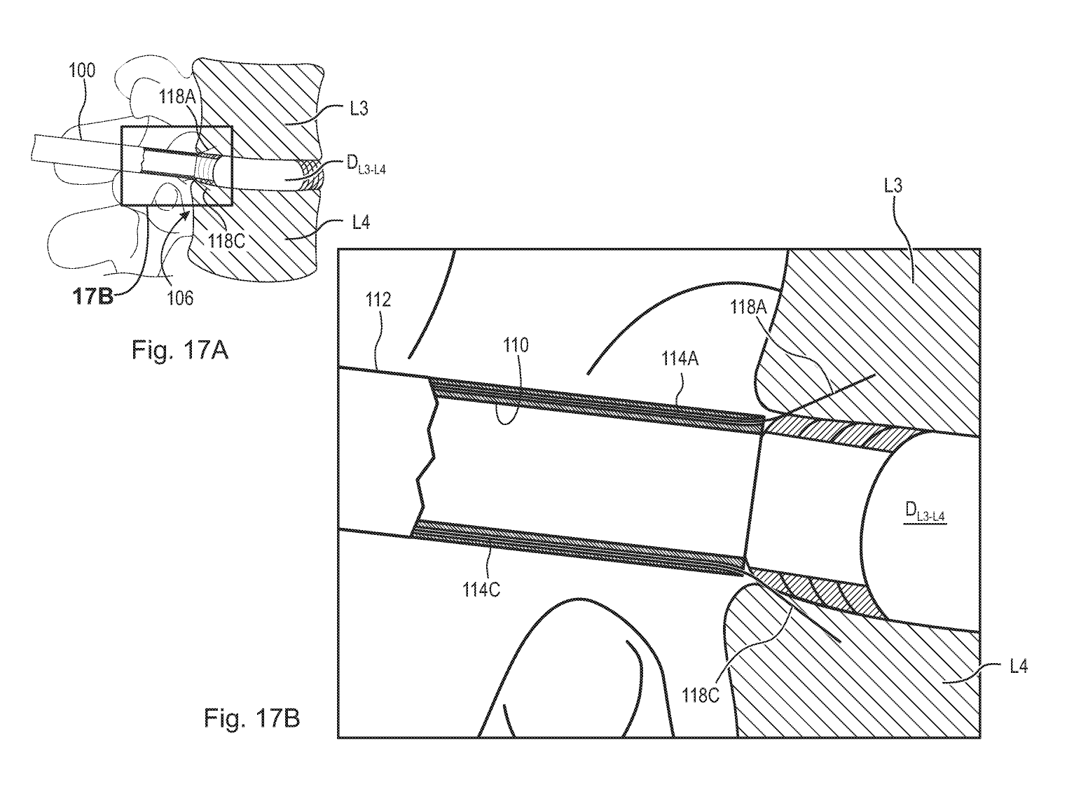

FIG. 16A illustrates a partial cross-sectional view of endoscopy sheath 100 proximate to disc D_{L3-L4} with anchoring wire 118A introduced into vertebra L3. FIG. 16B is an enlarged view of box 16B in FIG. 16A illustrating endoscopy sheath 100 proximate to disc D_{L3-L4} with anchoring wire 118A introduced into vertebra L3. FIG. 17A illustrates a partial cross-sectional view of endoscopy sheath 100 proximate to disc D_{L3-L4} with anchoring wires 118A and 118C introduced into vertebrae L3 and L4, respectively. During surgery, it may be desirable to use more than one anchoring wire, e.g., anchoring wires 118A and 118C, so as to limit the maximum range of pivotable motion of endoscopy sheath 100, while increasing the stability of endoscopy sheath 100. Although only two anchoring wires are shown in FIG. 17A, it should be appreciated that any combination of wires 118A-118F can be utilized to increase stability whilst lowering maximum range of motion of endoscopy sheath 100. FIG. 17B is an enlarged view of box 17B in FIG. 17A illustrating endoscopy sheath 100 proximate to disc D_{L3-L4} with anchoring wires 118A and 118C introduced into vertebrae L3 and L4, respectively.

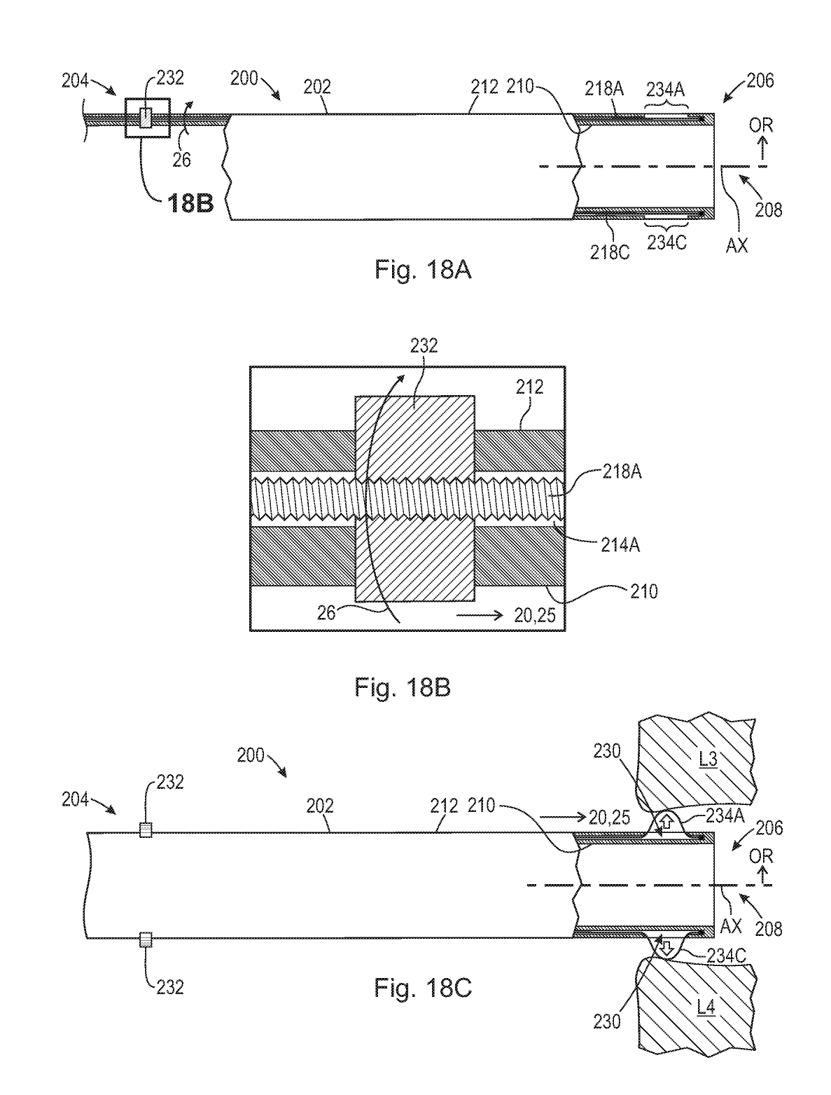

FIG. 18A is a partial cross-sectional side view of endoscopy sheath 200 having knurled wheel 232 and expandable anchor wires 218A and 218C having first deformable sections 234A and 234C in a retracted state. In this example embodiment, endoscopy sheath 200 comprises a tubular member 202. Tubular member 202 comprises a proximate end 204, a distal end 206, a through-bore 208, an inner surface 210, and an outer surface 212. Through-bore 208 is operatively arranged to receive, and slidingly engage with, endoscope 30. Inner surface 210 refers to the inner circumferential surface created by through-bore 208, and outer surface 212 refers to the outer circumferential surface of tubular member 202. Further, tubular member 202 comprises through-bore 208 which is arranged about and concentric with, imaginary axis AX. Between inner surface 210 and outer surface 212 there are channels 214A-214F (only 214A and 214C are shown). It should be appreciated that, although two (2) opposing channels are shown, any number of channels in any circumferentially spaced orientation can be utilized. Channels 214A-214F begin at proximate end 204 and terminate at distal end 206. In this example embodiment, expandable anchoring wire 218A comprises a threaded body and first deformable section 234A, and endoscopy sheath 200 comprises first aperture 230. During surgery, a surgeon or assistant can insert expandable anchoring wire 218A into channel 214A and proceed to apply rotational force 26 to knurled wheel 232. Expandable anchoring wires 218A-218F (only 218A and 218C are shown) each comprise first deformable sections 234A-234F (only 234A and 234C are shown), respectively. First deformable sections 234A-234F are made of any suitable material that can deform under pressure and resists breaking, e.g., a strip of stainless steel.

FIG. 18B is an enlarged view of box 18B in FIG. 18A illustrating knurled wheel 232 and its interaction with expandable anchoring wire 218A. Knurled wheel 232 operatively engages with the threaded body of expandable anchoring wire 218A imparting compressive force 25 onto anchoring wire 218A toward distal end 206 of endoscopy sheath 200.

FIG. 18C is a partial cross-sectional view of endoscopy sheath 200 having knurled wheel 232 and expandable anchor wires 218A and 218C having first deformable sections 234A and 234C in an expanded state. When compressive force 25 is applied to expandable anchoring wires 218A and 218C, first deformable sections 234A and 234C deform in outward radial direction OR, expanding out of first aperture 230. When sufficient force is applied such that first deformable sections 234A and 234C deform but do not break, first deformable sections 234A and 234C of expandable anchoring wires 218A and 218C contact vertebrae L3 and L4, respectively, holding endoscopy sheath 200 in place.

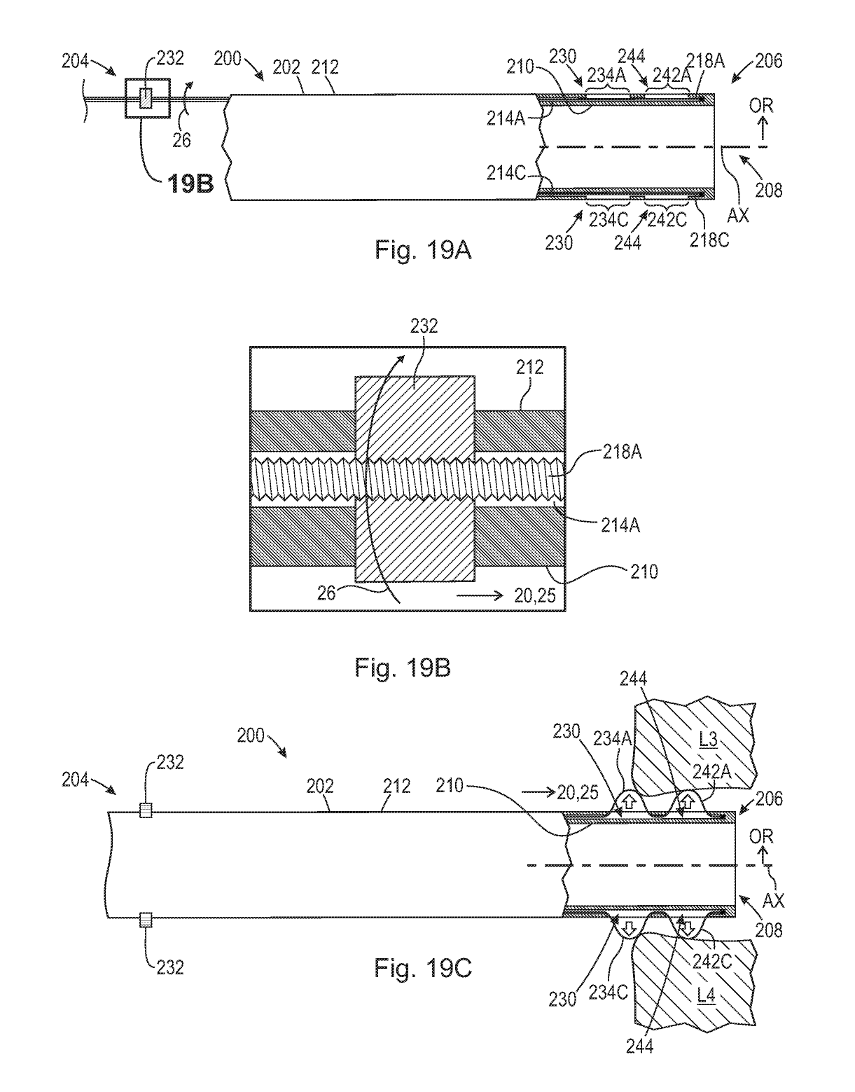

FIGS. 19A-19C illustrate endoscopy sheath 200 having knurled wheel 232 and expandable anchoring wires 218A and 218C with first deformable sections 234A and 234 C; and, second deformable sections 242A and 242C in a retracted state. This example embodiment is substantially similar to the embodiment illustrated in FIGS. 18A-18C except that this embodiment comprises first deformable sections 234A and 234C and first aperture 230, as well as second deformable sections 242A and 242C, first aperture 230 and a second aperture 244. When compressive force 25 is applied to expandable anchoring wires 218A and 218C, first deformable section 234A and second deformable section 242A, deform in outward radial direction OR, expanding out of first aperture 230 and second aperture 244, respectively. In the expanded state, illustrated in FIG. 19C, both first deformable section 234A and second deformable section 242A contact and engage with vertebrae L3 and L4, respectively, holding endoscopy sheath 200 in place.

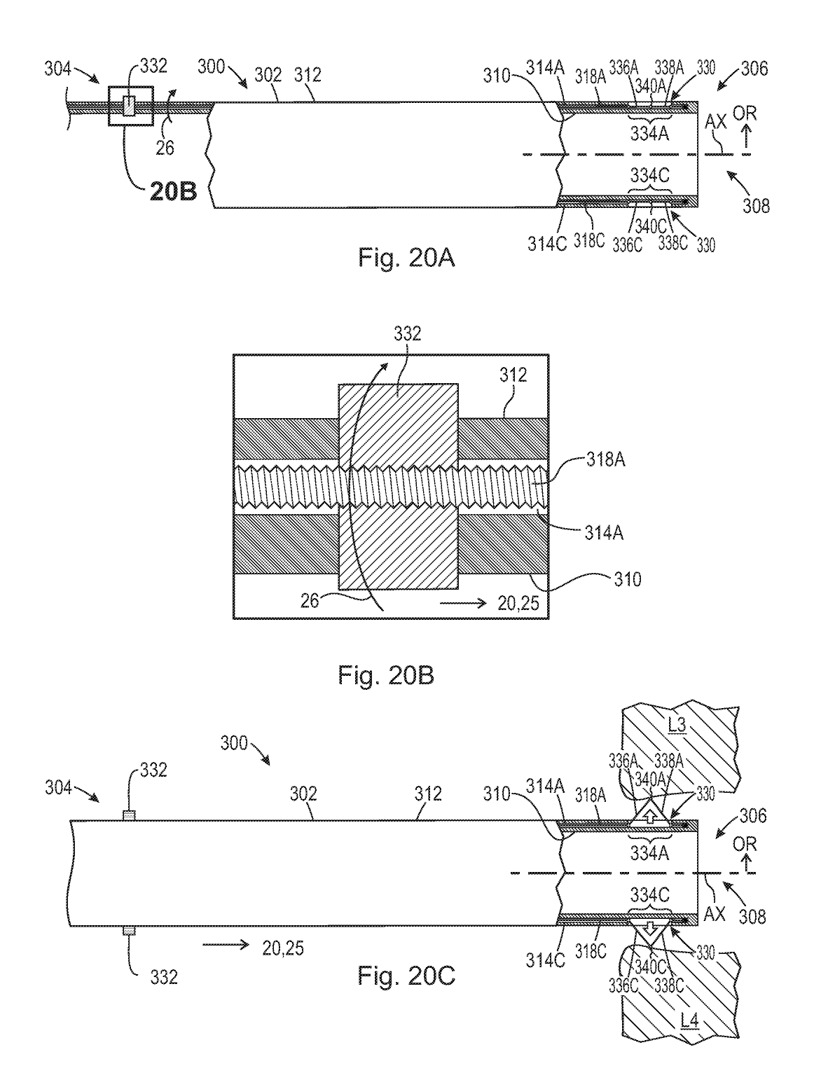

FIG. 20A is a partial cross-sectional view of the endoscopy sheath 300 having knurled wheel 332 and expandable anchor wires 318A and 318C having first pivotable sections 334A-334F (only 334A and 334C are shown) in a retracted state. Each of first pivotable sections 334A-334F comprise first sections 336A-336F (only 336A and 336C are shown), second sections 338A-338F (only 338A and 338C are shown), and first hinges 340A-340F (only 340A and 340C are shown). In this example embodiment, endoscopy sheath 300 comprises a tubular member 302. Tubular member 302 comprises a proximate end 304, a distal end 306, a through-bore 308, an inner surface 310, and an outer surface 312. Through-bore 308 is operatively arranged to receive, and slidingly engage with, endoscope 30. Inner surface 310 refers to the inner circumferential surface created by through-bore 308, and outer surface 312 refers to the outer circumferential surface of tubular member 302. Further, tubular member 302 comprises through-bore 308 which is arranged about and concentric with, imaginary axis AX. Between inner surface 310 and outer surface 312 there are channels 314A-314F. It should be appreciated that, although two (2) opposing channels are shown, any number of channels in any circumferentially spaced orientation can be utilized. Channels 314A-314F (only 314A and 314C are shown) begin at proximate end 304 and terminate at distal end 306. In this example embodiment, endoscopy sheath 300 comprises aperture 330. During surgery, a surgeon or assistant can insert expandable anchoring wire 318A into channel 314A and proceed to apply rotational force 26 to knurled wheel 332.

FIG. 20B is an enlarged view of box 20B in FIG. 20A illustrating knurled wheel 332 and its interaction with expandable anchoring wire 318A. Knurled wheel 332 operatively engages with the threaded body of expandable anchoring wire 318A imparting compressive force 25 onto anchoring wire 318A toward distal end 306 of endoscopy sheath 300.

FIG. 20C is a partial cross-sectional view of the endoscopy sheath 300 having knurled wheel 332 and expandable anchor wires 318A and 318C having first sections 336A and 336C; second sections 338A and 338C; and, first hinges 340A and 340C, respectively, in an expanded state. When compressive force 25 is applied to expandable anchoring wires 318A and 318C, first hinges 340A and 340C pivot in outward radial direction OR, allowing first sections 336A and 336C; second sections 338A and 338C; and, first hinges 340A and 340C to protrude out of aperture 330 and engage with vertebrae L3 and L4, respectively, holding endoscopy sheath 300 in place.

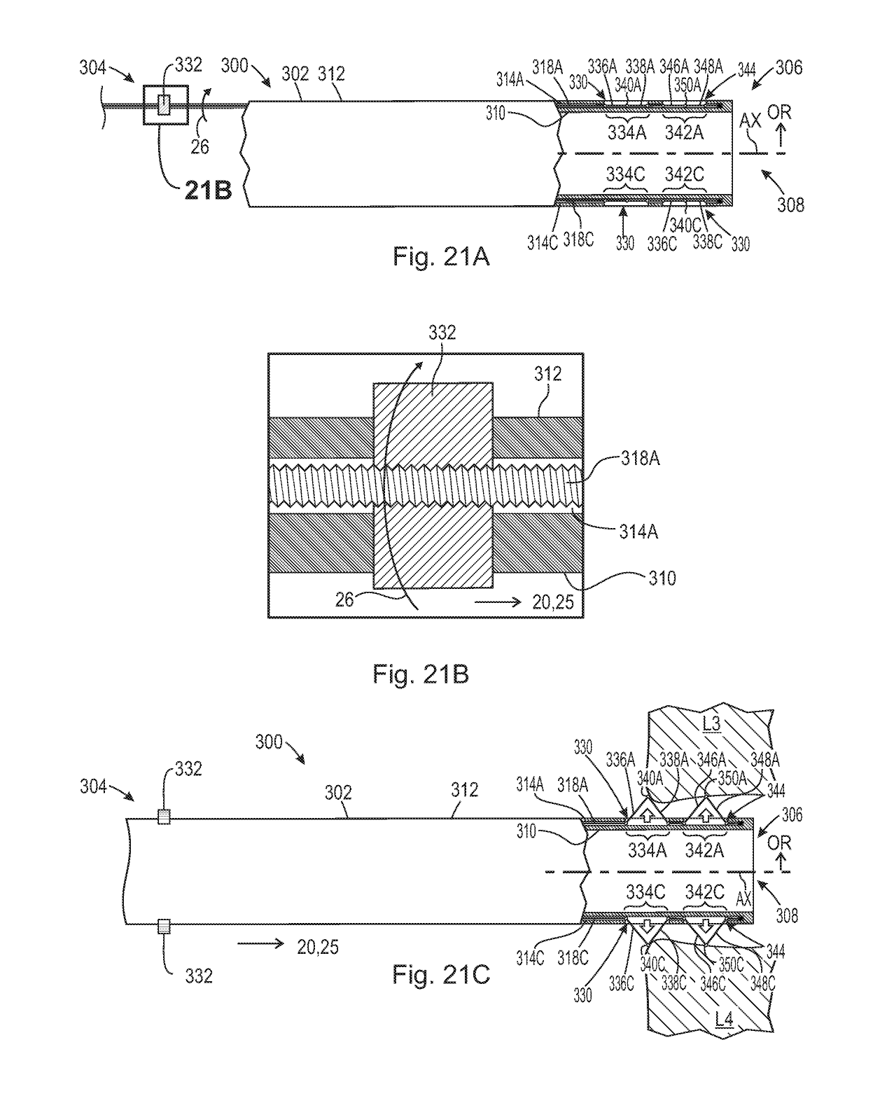

FIGS. 21A-21C illustrate endoscopy sheath 300 having knurled wheel 332 and first pivotable sections 334A and 334C, and second pivotable sections 342A and 342C in a retracted state. This example embodiment is substantially similar to the embodiment illustrated in FIGS. 20A-20C except that this embodiment illustrates first pivotable sections 334A and 334C, and second pivotable sections 342A and 342C, first aperture 330 and second aperture 344. Each of first pivotable sections 334A-334F (only 334A and 334C are shown) comprise first sections 336A-336F (only 336A and 336C are shown), second sections 338A-338F (only 348A and 348C are shown), and first hinges 340A-340F (only 340A and 340C are shown). Each of second pivotable sections 342A-342F (only 342A and 342F are shown) comprises third sections 346A-346F (only 346A and 346C are shown), fourth section 348A-348F (only 348A and 348C are shown), and second hinges 350A-350F (only 350A and 350C are shown). When compressive force 25 is applied to expandable anchoring wires 318A and 318C, first hinge 340A and second hinge 350A pivot in outward radial direction OR, allowing first section 336A, second section 338A, and first hinge 340A to protrude out of first aperture 330; and, third section 346A, fourth section 348A, and second hinge 350A to protrude out of second aperture 344. In its expanded state, illustrated in FIG. 21C, first hinge 340A and second hinge 350A contact and engage with adjacent vertebrae, i.e., first hinge 340A and second hinge 350A of expandable anchoring wire 318A both contact vertebra L3; and, first hinge 340C and second hinge 350C of expandable anchoring wire 318C both contact vertebra L4.

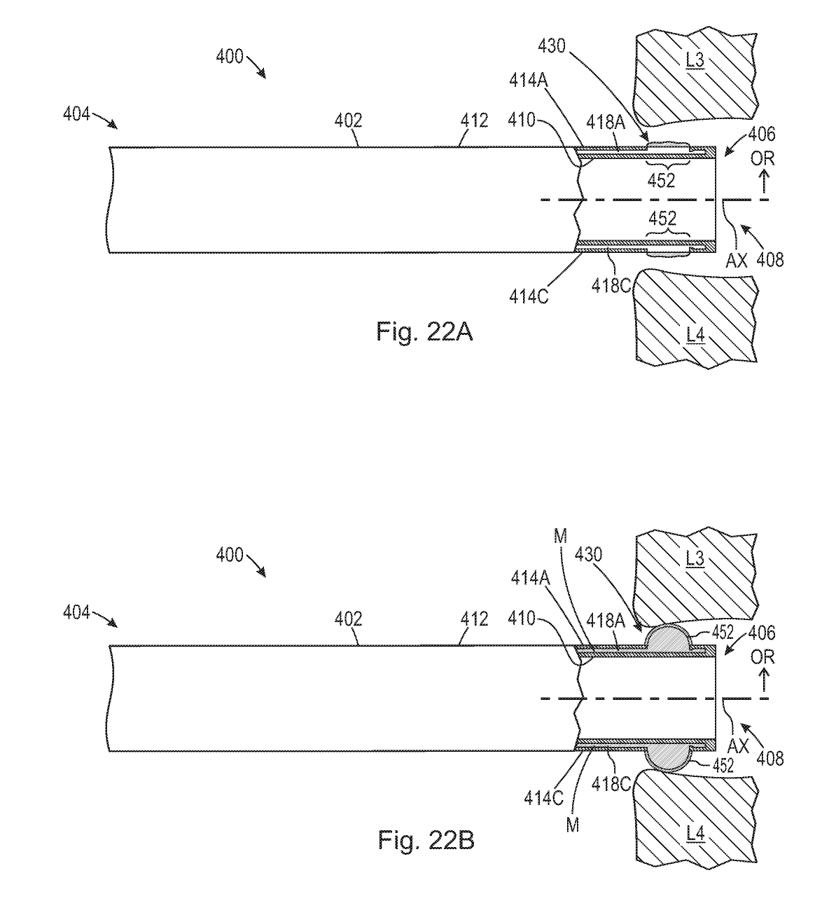

FIG. 22A is a partial cross-sectional view of endoscopy sheath 400 having first inflatable balloon collar 452 in a non-inflated state. In this example embodiment, endoscopy sheath 400 comprises a tubular member 402. Tubular member 402 comprises a proximate end 404, a distal end 406, a through-bore 408, an inner surface 410, and an outer surface 412 and first aperture 430. Through-bore 408 is operatively arranged to receive and slidingly engage with endoscope 30. Inner surface 410 refers to the inner circumferential surface created by through-bore 408, and outer surface 412 refers to the outer circumferential surface of tubular member 402. Further, tubular member 402 comprises axis AX, which travels through, and is concentric with, through-bore 408. Between inner surface 410 and outer surface 412 there are channels 414A-414F. It should be appreciated that, although two (2) opposing channels are shown, any number of channels in any circumferentially spaced orientation can be utilized. Each of channels 414A-414F (only 414A and 414C are shown) begin at proximate end 404 and terminate proximate to distal end 406 into first inflatable balloon collar 452. Channels 414A-414F act as a conduit into which air, water, or saline is injected or insufflated so as to allow for variable expansion of first inflatable balloon collar 452. First inflatable balloon collar 452 is fixedly secured to outer surface 412 covering first aperture 430. First balloon collar 452 could be affixed using any suitable adhesive that would keep first balloon collar 452 fixedly secured to outer surface 412 of tubular member 402 during expansion. Alternatively, any device suitable to encompass the circumference of tubular member 402 and provide sufficient force to keep first balloon collar 452 fixedly secured to tubular member 402 can be used, e.g., a ring clamp, hose clamp, zip-ties, etc. Although not shown, the surface of first balloon collar 452 could be rough or corrugated to aid in increasing the static coefficient of friction required to stabilize the distal end 406 of endoscopy sheath 400 and prevent it from slipping. Further, first balloon collar 452 could be made from a latex or peek material.

FIG. 22B is a partial cross-sectional view of endoscopy sheath 400 having first inflatable balloon collar 452 in an inflated state. In the inflated state first balloon collar 452 is full of air, water, saline, or equivalent material, such that first balloon collar 452 protrudes through first aperture 430. In the inflated state, first balloon collar 452 is in contact with, and frictionally engages, vertebrae L3 and L4, holding endoscopy sheath 400 in place.

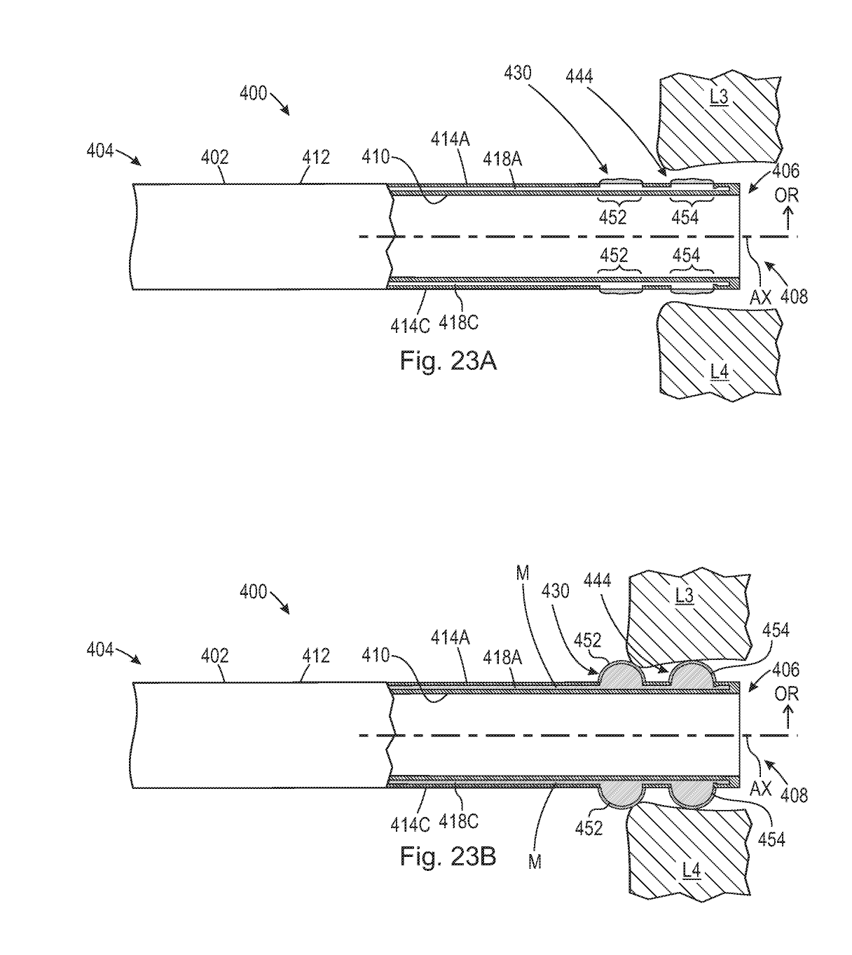

FIG. 23A is a partial cross-sectional view of endoscopy sheath 400 having first inflatable balloon collar 452 and second inflatable balloon collar 454 in a non-inflated state. This example embodiment is substantially similar to the embodiment illustrated in FIGS. 22A-22B except that in this embodiment, each of channels 414A-414F terminate into first inflatable balloon collar 452 and second inflatable balloon collar 454. Additionally, tubular member 402 comprises first aperture 430 and second aperture 444.

FIG. 23B is a partial cross-sectional view of endoscopy sheath 400 having first inflatable balloon collar 452 and second inflatable balloon collar 454 in an inflated state. In the inflated state, first inflatable balloon collar 452 and second inflatable balloon collar 454 are full of air, water, saline, or equivalent material such that, first inflatable balloon collar 452 and second inflatable balloon collar 454 protrude through first aperture 430 and second aperture 444, respectively. In the inflated state, first inflatable balloon collar 452 and second inflatable balloon collar 454 are in contact with, and frictionally engage, vertebrae L3 and L4, holding endoscopy sheath 400 in place. Although not shown, the surface of first balloon collar 452 and the surface of second balloon collar 454 could be rough or corrugated to aid in increasing the static coefficient of friction required to stabilize the distal end 406 of endoscopy sheath 400 and prevent it from slipping. Further, second balloon collar could be made from a latex or peek material.

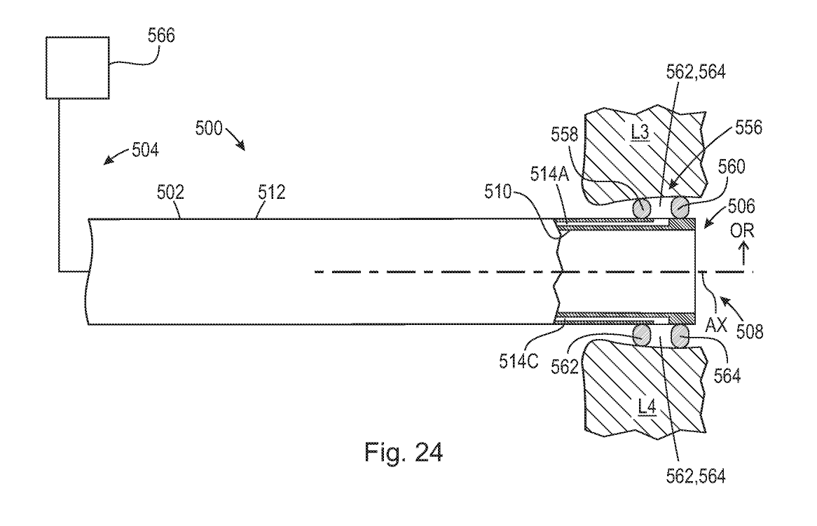

FIG. 24 is a partial cross-sectional view of endoscopy sheath 500 having a vacuum collar 556. Vacuum collar 556 comprises first sealing member 558 and second sealing member 560. In this example embodiment, endoscopy sheath 500 comprises a tubular member 502. Tubular member 502 comprises a proximate end 504, a distal end 506, a through-bore 508, an inner surface 510, and an outer surface 512. Through-bore 508 is operatively arranged to receive and slidingly engage with endoscope 30. Inner surface 510 refers to the inner circumferential surface created by through-bore 508, and outer surface 512 refers to the outer circumferential surface of tubular member 502. Further, tubular member 502 comprises axis AX, which travels through, and is concentric with, through-bore 508. Between inner surface 510 and outer surface 512 there are channels 514A-514F (only 514A and 514C are shown). It should be appreciated that, although only two (2) channels are shown, any number of channels in any circumferentially spaced orientation can be utilized. Channels 514A-514F begin at proximate end 504 and terminate proximate at distal end 506 into cavity 562. Channels 514A-514F act as a conduit through which negative pressure 564 can be applied by vacuum source 566. During surgery, a surgeon or assistant can use vacuum source 566, e.g., a wall suction or equivalent, to create negative pressure 564 throughout channels 514A-514F as well as cavity 562. First sealing member 558 and second sealing member 560 are operatively arranged to engage with both vertebrae L3 and L4 to prevent pressure or air from escaping cavity 562. This creates a vacuum or suction-type adherence to the surrounding vertebrae and keeps distal end 506 of endoscopy sheath 500 in place.

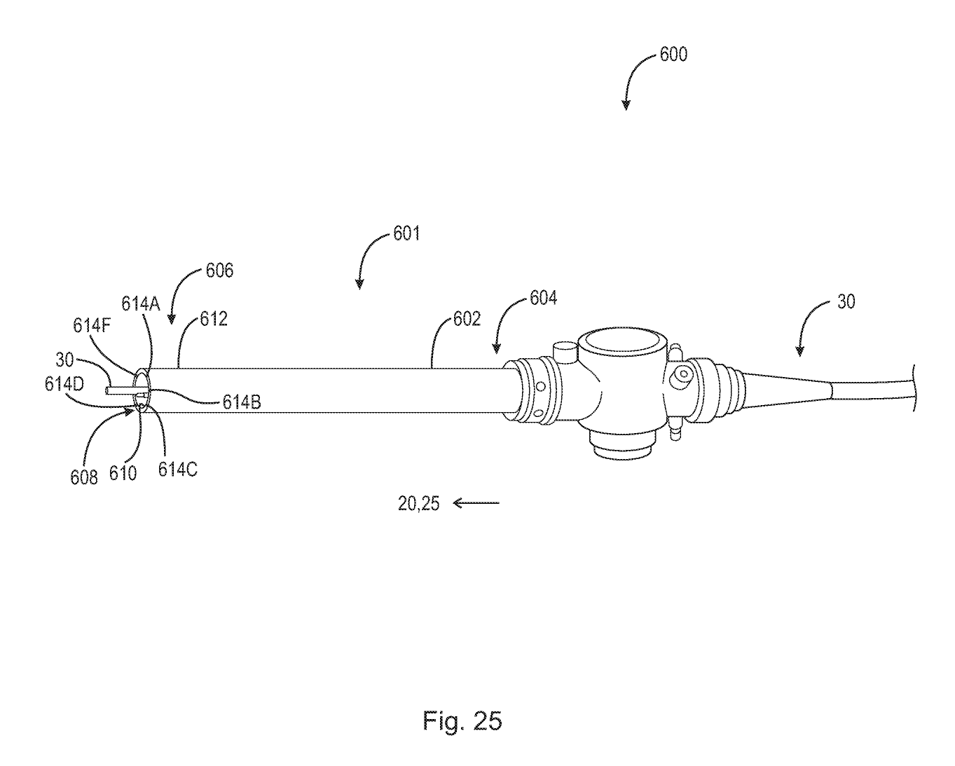

FIG. 25 is a side perspective view of endoscope assembly 600 having endoscope 30 operatively arranged within an endoscopy sheath 601. Endoscopy sheath 601 comprises a tubular member 602. Tubular member 602 comprises proximate end 604, distal end 606, a through-bore 608, an inner surface 610, and an outer surface 612. Through-bore 608 is operatively arranged to receive and slidingly engage with endoscope 30. Inner surface 610 refers to the inner circumferential surface created by through-bore 608, and outer surface 612 refers to the outer circumferential surface of tubular member 602. Further, tubular member 602 comprises axis AX, which travels through, and is concentric with, through-bore 608. Between inner surface 610 and outer surface 612 there are channels 614A-614F. It should be appreciated that, although six (6) equally spaced channels are shown, any number of channels in any circumferentially spaced orientation can be utilized. Channels 614A-614F begin at proximate end 604 and terminate at distal end 606. This example embodiment illustrates an endoscope 30 after it has been inserted within endoscope 601. During surgery, a surgeon or assistant begins by positioning distal end 606 of endoscopy sheath 601 close to the site where surgery will be taking place, e.g., close to disc D_{L3-L4}. The surgeon or assistant can then impart concussive force 20 or compressive force 25 onto anchoring wires 618A-618F (not shown in FIG. 25) to secure distal end 606 within tissue 46. The surgeon or assistant can now insert endoscope 30 into, and translate through, through-bore 608 until endoscope 30 is in position for surgery.

Thus it is seen that the objects of the invention are efficiently obtained, although changes and modifications to the invention should be readily apparent to those having ordinary skill in the art, which changes would not depart from the spirit and scope of the invention as claimed.

### LIST OF REFERENCE NUMBERS

10 Spinal column
AX Axis
C1-C7 Cervical vertebrae
T1-T9 Thoracic vertebrae
L1-L5 Lumbar vertebrae
S Sacrum
C Coccyx
D_{L1-L2} Disc
D_{L2-L3} Disc
D_{L3-L4} Disc
D_{L4-L5} Disc
F Facet
FJ Facet joint
SP Spinous process
TP Transverse process
IF Intervertebral foramen
A Annulus
N Nucleus
M First material
OR Outward radial direction
IR Inward radial direction
20 Concussive force
25 Compressive force
26 Rotational force
30 Endoscope
31 Light guide connector
32 Light guide tube
33 Control body
34 Insertion tube
40 Surgeon
41 Monitor
45 Patient
46 Tissue
50 Anchoring wire
52 Unthreaded body
54 Unthreaded tip
60 Anchoring wire
62 Unthreaded body
64 Threaded tip
70 Anchoring wire
72 Threaded body
74 Threaded tip
100 Endoscopy sheath
102 Tubular member
104 Proximate end
106 Distal End
108 Through-bore
110 Inner surface
112 Outer surface
114A Channel
114B Channel
114C Channel
114D Channel
114E Channel
114F Channel
115A Semi-open external channel
115B Semi-open external channel
115C Semi-open external channel
115D Semi-open external channel
115E Semi-open external channel
115F Semi-open external channel
116A Ramp
116B Ramp
116C Ramp
116D Ramp
116E Ramp
116F Ramp
117A Semi-open external removable channel
117B Semi-open external removable channels
117C Semi-open external removable channel
117D Semi-open external removable channel
117E Semi-open external removable channel
117F Semi-open external removable channel
118A Anchoring wire
118B Anchoring wire
118C Anchoring wire
118D Anchoring wire
118E Anchoring wire
118F Anchoring wire
119A Semi-open internal channel
119B Semi-open internal channels
119C Semi-open internal channel
119D Semi-open internal channel
119E Semi-open internal channel
119F Semi-open internal channel
120A Longitudinal space
120B Longitudinal space
120C Longitudinal space
120D Longitudinal space
120E Longitudinal space
120F Longitudinal space
122A Recess
122B Recess
122C Recess
122D Recess
122E Recess
122F Recess
124 Impeller
125 Pivot
126 Handle
127 First ring
128 Lever
129 Second ring
200 Endoscopy sheath
202 Tubular member
204 Proximate end
206 Distal End
208 Through-bore
210 Inner surface
212 Outer surface
214A Channel
214B Channel
214C Channel
214D Channel
214E Channel
214F Channel
218A Expandable anchoring wire
218B Expandable anchoring wire
218C Expandable anchoring wire
218D Expandable anchoring wire
218E Expandable anchoring wire
218F Expandable anchoring wire
230 First aperture
232 Knurled wheel
234A First deformable section
234B First deformable section
234C First deformable section
234D First deformable section
234E First deformable section
234F First deformable section
242A Second deformable section
242B Second deformable section
242C Second deformable section
242D Second deformable section
242E Second deformable section
242F Second deformable section
244 Second aperture
300 Endoscopy sheath
302 Tubular member
304 Proximate end
306 Distal End
308 Through-bore
310 Inner surface
312 Outer surface
314A Channel
314B Channel
314C Channel
314D Channel
314E Channel
314F Channel
318A Expandable anchoring wire
318B Expandable anchoring wire
318C Expandable anchoring wire
318D Expandable anchoring wire
318E Expandable anchoring wire
318F Expandable anchoring wire
330 First aperture
332 Knurled wheel
334A First pivotable section
334B First pivotable section
334C First pivotable section
334D First pivotable section
334E First pivotable section
334F First pivotable section
336A First section
336B First section
336C First section
336D First section
336E First section
336F First section
338A Second section
338B Second section
338C Second section
338D Second section
338E Second section
338F Second section
340A First hinge
340B First hinge
340C First hinge
340D First hinge
340E First hinge
340F First hinge
342A Second pivotable section
342B Second pivotable section
342C Second pivotable section
342D Second pivotable section
342E Second pivotable section
342F Second pivotable section
344 Second aperture
346A Third section
346B Third section
346C Third section
346D Third section
346E Third section
346F Third section
348A Fourth section
348B Fourth section
348C Fourth section
348D Fourth section
348E Fourth section
348F Fourth section
350A Second hinge
350B Second hinge
350C Second hinge
350D Second hinge
350E Second hinge
350F Second hinge
400 Endoscopy sheath
402 Tubular member
404 Proximate end
406 Distal End
408 Through-bore
410 Inner surface
412 Outer surface
414A Channel
414B Channel
414C Channel
414D Channel
414E Channel
414F Channel
430 First aperture
444 Second aperture
452 First inflatable balloon collar
454 Second inflatable balloon collar
500 Endoscopy sheath
502 Tubular member
504 Proximate end
506 Distal End
508 Through-bore
510 Inner surface
512 Outer surface
514A Channel
514B Channel
514C Channel
514D Channel
514E Channel
514F Channel
530 First aperture
544 Second aperture
556 Vacuum collar
558 First sealing member
560 Second sealing member
562 Cavity
564 Negative pressure
566 Vacuum source
600 Endoscope assembly
601 Endoscopy sheath
602 Tubular member
604 Proximate end
606 Distal end
608 Through-bore
610 Inner surface
612 Outer surface
614A Channel
614B Channel
614C Channel
614D Channel
614E Channel
614F Channel

I claim:

1. A self-anchoring endoscopy sheath, comprising:
a tubular member comprising:
a proximate end;
a distal end;
a through-bore;
an inner surface; and,
an outer surface;
at least one anchoring wire having a body and a tip; and,
at least one channel arranged between the inner surface and the outer surface, the at least one channel operatively arranged to receive and slidingly engage with the at least one anchoring wire;
wherein, the tip of the anchoring wire is operatively arranged to engage with a tissue and the through-bore is operatively arranged to receive an endoscope.

2. The self-anchoring endoscopy sheath recited in claim 1, wherein the tubular member further comprises at least one ramp arranged at the distal end of the tubular member and within the at least one channel, the at least one ramp operatively arranged to divert the at least one anchor wire in an outward radial direction.

3. The self-anchoring endoscopy sheath recited in claim 1, wherein the at least one channel is arranged on the outer surface of the tubular member, the at least one channel further comprising a longitudinal space.

4. The self-anchoring endoscopy sheath recited in claim 3, wherein the at least one channel is arranged on the outer surface of the tubular member and is removable, the at least one channel further comprising a longitudinal space.

5. The self-anchoring endoscopy sheath recited in claim 1 wherein the outer surface comprises at least one longitudinal space radially outward of the at least one channel.

6. The self-anchoring endoscopy sheath recited in claim 1, wherein the body and the tip of the at least one anchoring wire is unthreaded.

7. The self-anchoring endoscopy sheath recited in claim 1, wherein the body of the at least one anchoring wire is unthreaded and the tip is threaded.

8. The self-anchoring endoscopy sheath recited in claim 1, wherein the body and the tip of the at least one anchoring wire are threaded.

9. The self-anchoring endoscopy sheath recited in claim 1, further comprising a knurled wheel arranged at the proximate end of the tubular member, the knurled wheel operatively arranged to impart a compressive force to the body of the at least one anchoring wire.

10. The self-anchoring endoscopy sheath recited in claim 1, further comprising a impeller comprising:
a handle;
a lever pivotably secured to the handle at a pivot;
a first ring pivotably secured to the handle; and,
a second ring pivotably secured to the lever,
wherein the second ring is fixedly secured to the at least one anchoring wire and arranged to impart a compressive force to the body of the at least one anchoring wire.

11. A self-anchoring endoscopy sheath, comprising:
a tubular member comprising:
a proximate end;
a distal end;
a through-bore;
an inner surface; and,
an outer surface;
a first expandable anchoring wire; and,
at least one channel arranged between the inner surface and the outer surface, the at least one channel operatively arranged to receive the first expandable anchoring wire,
wherein the first expandable anchoring wire is operatively arranged to engage with a tissue and the through-bore is operatively arranged to receive an endoscope.

12. The self-anchoring endoscopy sheath recited in claim 11, wherein the first expandable anchoring wire further comprises a pivotable section comprising:
a first section;
a second section; and,
a first hinge operatively arranged between the first and second section.

13. The self-anchoring endoscopy sheath recited in claim 12, wherein the tubular member further comprises a first aperture, operatively arranged to receive the first section, the second section, and the first hinge.

14. The self-anchoring endoscopy sheath recited in claim 11, wherein the tubular member further comprises a first aperture operatively arranged to receive a first material, and a first balloon collar fixedly secured to the tubular member and arranged to receive the first material.

15. The self-anchoring endoscopy sheath recited in claim 14, wherein the tubular member further comprises a second aperture operatively arranged to receive the first material, and a second balloon collar fixedly secured to the tubular member and operatively arranged to receive the first material.

16. The self-anchoring endoscopy sheath recited in claim 11, wherein the first expandable anchoring wire comprises:
a first section;
a second section;
a third section;
a fourth section;
a first hinge operatively arranged between the first and second sections; and,
a second hinge operatively arranged between the third and fourth sections.

17. The self-anchoring endoscopy sheath recited in claim 16, wherein the tubular member further comprises a first aperture operatively arranged to receive the first section, second section, and the first hinge, and a second aperture operatively arranged to receive the third section, fourth section, and the second hinge.

18. The self-anchoring endoscopy sheath recited in claim 11, wherein the first expandable anchor wire comprises a first deformable section, the first deformable section is deformable in an outward radial direction in response to a compressive force applied to the first expandable anchor wire.

19. A self-anchoring endoscopy sheath, comprising:
a tubular member comprising:
a proximate end;
a distal end;
a through-bore;
an inner surface; and,
an outer surface;
a first sealing member;
a second sealing member;
a cavity, arranged between the first and second sealing members;
at least one channel arranged to between the inner surface and the outer surface, the at least one channel operatively arranged to connect a vacuum source to the cavity;
wherein the vacuum source generates a negative pressure within the channel and the cavity, securing the tubular member to a tissue via suction.

20. The self-anchoring endoscopy sheath recited in claim 19, wherein the first sealing member and second sealing member are disposed circumferentially around the tubular member.

21. An endoscope assembly comprising:
an endoscope; and,
a self-anchoring endoscopy sheath, comprising:
a tubular member comprising:
a proximate end;
a distal end;
a through-bore;
an inner surface; and,
an outer surface;
at least one anchoring wire having a body and a tip; and,
at least one channel arranged between the inner surface and the outer surface, the at least one channel operatively arranged to receive and slidingly engage with the at least one anchoring wire;
wherein, the tip of the anchoring wire is operatively arranged to engage with a tissue and the through-bore is operatively arranged to receive the endoscope.
